(19) United States
(12) Patent Application Publication
(10) Pub. No.: US 2025/0285766 A1
(43) Pub. Date: Sep. 11, 2025

(54) CAUSAL DISCOVERY AND INFERENCING FOR MEDICAL SYSTEMS

(71) Applicant: Auris Health, Inc., Santa Clara, CA (US)

(72) Inventors: Lauren Elizabeth Friend, San Francisco, CA (US); Ryan David Kappedal, Olympia, WA (US)

(73) Assignee: Auris Health, Inc., Santa Clara, CA (US)

(21) Appl. No.: 19/074,278

(22) Filed: Mar. 7, 2025

Related U.S. Application Data
(60) Provisional application No. 63/563,901, filed on Mar. 11, 2024.

(51) Int. Cl. G16H 50/30; G06F 16/28; G16H 10/20; G16H 40/60

(52) U.S. Cl. CPC G16H 50/30; G06F 16/288; G16H 10/20; G16H 40/60

(57) ABSTRACT

This disclosure provides methods, devices, and systems for causal inferencing. The present implementations more specifically relate to determining causal relationships between clinical metrics associated with a procedure performed, at least in part, by a medical system. In some aspects, an inferencing system may receive case data or telemetry generated by the medical system and determine a set of clinical metrics associated with the procedure based on the case data. The inferencing system further maps the set of clinic metrics to a directed acyclic graph (DAG) based on one or more casual relationships between the various clinical metrics. For example, the DAG may indicate which of the clinical metrics are causally related, including which clinical metric has a causal effect on the other. The inferencing system further generates one or more inferences associated with the medical system based on the DAG and/or data generation information associated with the clinical metrics.

### CROSS-REFERENCE TO RELATED APPLICATIONS

This application claims priority and benefit under 35 U.S.C. § 119 (e) to U.S. Provisional Patent Application No. 63/563,901, filed Mar. 11, 2024, which is incorporated herein by reference in its entirety.

### TECHNICAL FIELD

This disclosure relates generally to medical systems, and specifically to causal discovery and inferencing for medical systems.

### DESCRIPTION OF RELATED ART

Many medical procedures, such as laparoscopy, ureteroscopy, or percutaneous nephrolithotomy (PCNL), involve a series of complex steps that require careful movement and positioning of medical tools or instruments inside a patient's body. For example, to remove urinary stones from the bladder and ureter, surgeons can insert a ureteroscope into the urinary tract through the urethra. A ureteroscope includes an endoscope at its distal end configured to enable visualization of the urinary tract. Generally, during a percutaneous access procedure (such as PCNL), the ureteroscope can be used to designate or “tag” a target location for a needle to access the kidney percutaneously. The physician drives the needle into the patient, through the target location, and uses another medical instrument (which may be in conjunction with the needle) to extract the kidney stone from the patient via the percutaneous access point. The success or failure of such medical procedures often depends on various factors, including the physician's skill, the patient's anatomy, and the quality of any tools or equipment the physician uses to perform the procedure.

Medical procedures are often recorded or documented in “case logs,” which can then be analyzed for training purposes and medical research. For example, when a medical procedure is unsuccessful (such as where a physician fails to remove a kidney stone or to even gain percutaneous access to a patient's kidney for a PCNL procedure), the physician may review the associated case log to determine how they can improve. However, raw case log data may not be suitable for evaluating the success of the medical procedure or the performance of the physician. The raw case log data also does not directly provide any interoperative insights or recommendations, such as identifying what caused the medical procedure (or any interoperative procedures) to succeed or fail. Thus, there is a need to provide greater insights about medical procedures using the data that is captured or recorded from the medical procedures.

### SUMMARY

This Summary is provided to introduce in a simplified form a selection of concepts that are further described below in the Detailed Description. This Summary is not intended to identify key features or essential features of the claimed subject matter, nor is it intended to limit the scope of the claimed subject matter.

One innovative aspect of the subject matter of this disclosure can be implemented in a computer-implemented method for causal inferencing. The method includes steps of receiving data associated with a procedure performed by a medical system; determining a plurality of clinical metrics associated with the procedure based at least in part on the received data; mapping the plurality of clinical metrics to a directed acyclic graph (DAG) based on a causal discovery operation that identifies one or more causal relationships between the plurality of clinical metrics; and generating one or more inferences associated with the medical system based at least in part on the DAG.

Another innovative aspect of the subject matter of this disclosure can be implemented in a causal inferencing system, including a processing system and a memory. The memory stores instructions that, when executed by the processing system, cause the causal inferencing system to receive data associated with a procedure performed by a medical system; determine a plurality of clinical metrics associated with the procedure based at least in part on the received data; map the plurality of clinical metrics to a DAG based on a causal discovery operation that identifies one or more causal relationships between the plurality of clinical metrics; and generate one or more inferences associated with the medical system based at least in part on the DAG.

### BRIEF DESCRIPTION OF THE DRAWINGS

The present implementations are illustrated by way of example and are not intended to be limited by the figures of the accompanying drawings.

FIG. 1 shows an example medical system, according to some implementations.

FIG. 2 shows a top view of the medical system of FIG. 1 configured to assist in inserting a scope into a patient.

FIG. 3 shows a top view of the medical system of FIG. 1 configured to navigate a scope within a patient.

FIG. 4 shows a top view of medical system of FIG. 1 configured to assist in inserting a needle into a patient.

FIG. 5 shows a block diagram of an example analytics engine, according to some implementations.

FIG. 6A shows a block diagram of an example many-to-one phase segmentation module, according to some implementations.

FIG. 6B shows a block diagram of an example many-to-many phase segmentation module, according to some implementations.

FIG. 6C shows a timing diagram depicting an example binary classification that can be performed by a segmentation module, according to some implementations.

FIG. 6D shows an example output of a segmentation module, according to some implementations.

FIG. 7 shows an illustrative flowchart depicting an example operation for evaluating phases in a percutaneous medical procedure, according to some implementations.

FIG. 8 shows an illustrative flowchart depicting an example operation for generating clinical metrics associated with a percutaneous medical procedure, according to some implementations.

FIG. 9 shows a block diagram of an example inferencing system, according to some implementations.

FIG. 10A shows an example chain that can be a component of a directed acyclic graph (DAG).

FIG. 10B shows an example collider that can be a component of a DAG.

FIG. 10C shows an example fork that can be a component of a DAG.

FIG. 11A shows an example fully connected graph of clinical metrics that can be provided as input to a causal discovery system.

FIG. 11B shows an example causal graph indicating various casual dependencies between the clinical metrics shown in FIG. 11A.

FIG. 11C shows an example DAG indicating various causal effects of the clinical metrics shown in FIG. 11A.

FIG. 12A shows another example fully connected graph of clinical metrics that can be provided as input to a causal discovery system, according to some implementations.

FIG. 12B shows another example causal graph indicating various casual dependencies between the clinical metrics shown in FIG. 12A.

FIG. 12C shows another example DAG indicating various causal effects of the clinical metrics shown in FIG. 12A.

FIG. 13 shows another example matrix representing the DAG depicted in FIG. 12C.

FIG. 14A shows another example fully connected graph of clinical metrics that can be provided as input to a causal discovery system, according to some implementations.

FIG. 14B shows another example causal graph indicating various casual dependencies between the clinical metrics shown in FIG. 14A.

FIG. 14C shows another example DAG indicating various causal effects of the clinical metrics shown in FIG. 14A.

FIG. 15 shows another example matrix representing the DAG depicted in FIG. 14C.

FIG. 16 shows a block diagram of an example causal inferencing system, according to some implementations.

FIG. 17 shows an illustrative flowchart depicting an example causal inferencing operation, according to some implementations.

### DETAILED DESCRIPTION

In the following description, numerous specific details are set forth such as examples of specific components, circuits, and processes to provide a thorough understanding of the present disclosure. The term “coupled” as used herein means connected directly to or connected through one or more intervening components or circuits. The terms “electronic system” and “electronic device” may be used interchangeably to refer to any system capable of electronically processing information. Also, in the following description and for purposes of explanation, specific nomenclature is set forth to provide a thorough understanding of the aspects of the disclosure. However, it will be apparent to one skilled in the art that these specific details may not be required to practice the example implementations. In other instances, well-known circuits and devices are shown in block diagram form to avoid obscuring the present disclosure. Some portions of the detailed descriptions which follow are presented in terms of procedures, logic blocks, processing and other symbolic representations of operations on data bits within a computer memory.

These descriptions and representations are the means used by those skilled in the data processing arts to most effectively convey the substance of their work to others skilled in the art. In the present disclosure, a procedure, logic block, process, or the like, is conceived to be a self-consistent sequence of steps or instructions leading to a desired result. The steps are those requiring physical manipulations of physical quantities. Usually, although not necessarily, these quantities take the form of electrical or magnetic signals capable of being stored, transferred, combined, compared, and otherwise manipulated in a computer system. It should be borne in mind, however, that all of these and similar terms are to be associated with the appropriate physical quantities and are merely convenient labels applied to these quantities.

Unless specifically stated otherwise as apparent from the following discussions, it is appreciated that throughout the present application, discussions utilizing the terms such as “accessing,” “receiving,” “sending,” “using,” “selecting,” “determining,” “normalizing,” “multiplying,” “averaging,” “monitoring,” “comparing,” “applying,” “updating,” “measuring,” “deriving” or the like, refer to the actions and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

Certain standard anatomical terms of location may be used herein to refer to the anatomy of animals, and namely humans, with respect to the example implementations. Although certain spatially relative terms, such as “outer,” “inner,” “upper,” “lower,” “below,” “above,” “vertical,” “horizontal,” “top,” “bottom,” and similar terms, are used herein to describe a spatial relationship of one element, device, or anatomical structure to another device, element, or anatomical structure, it is understood that these terms are used herein for ease of description to describe the positional relationship between elements and structures, as illustrated in the drawings. It should be understood that spatially relative terms are intended to encompass different orientations of the elements or structures, in use or operation, in addition to the orientations depicted in the drawings. For example, an element or structure described as “above” another element or structure may represent a position that is below or beside such other element or structure with respect to alternate orientations of the subject patient, element, or structure, and vice-versa. As used herein, the term “patient” may generally refer to humans, anatomical models, simulators, cadavers, and other living or non-living objects.

In the figures, a single block may be described as performing a function or functions; however, in actual practice, the function or functions performed by that block may be performed in a single component or across multiple components, or may be performed using hardware, using software, or using a combination of hardware and software. To clearly illustrate this interchangeability of hardware and software, various illustrative components, blocks, modules, circuits, and steps have been described below generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure. Also, the example systems or devices may include components other than those shown, including well-known components such as a processor, memory and the like.

The techniques described herein may be implemented in hardware, software, firmware, or any combination thereof, unless specifically described as being implemented in a specific manner. Any features described as modules or components may also be implemented together in an integrated logic device or separately as discrete but interoperable logic devices. If implemented in software, the techniques may be realized at least in part by a non-transitory processor-readable storage medium including instructions that, when executed, performs one or more of the methods described herein. The non-transitory processor-readable data storage medium may form part of a computer program product, which may include packaging materials.

The non-transitory processor-readable storage medium may comprise random access memory (RAM) such as synchronous dynamic random-access memory (SDRAM), read only memory (ROM), non-volatile random access memory (NVRAM), electrically erasable programmable read-only memory (EEPROM), FLASH memory, other known storage media, and the like. The techniques additionally, or alternatively, may be realized at least in part by a processor-readable communication medium that carries or communicates code in the form of instructions or data structures and that can be accessed, read, or executed by a computer or other processor.

The various illustrative logical blocks, modules, circuits and instructions described in connection with the implementations disclosed herein may be executed by one or more processors (or a processing system). The term “processor,” as used herein may refer to any general-purpose processor, special-purpose processor, conventional processor, controller, microcontroller, or state machine capable of executing scripts or instructions of one or more software programs stored in memory.

As described above, many medical procedures involve a series of complex steps that require careful movement and positioning of medical tools or instruments inside a patient's body. Some medical procedures can now be performed, at least in part, by a robotic system or apparatus, which can aid the physician in navigating or positioning such medical instruments. For example, to perform a robotically-assisted percutaneous nephrolithotomy (PCNL) procedure, a physician controls a robotic system to advance and navigate a medical instrument (such as a scope) from the urethra, through the bladder, up the ureter, and into the kidney where the kidney stone is located. The robotic system may include, or may be coupled to, one or more display devices that can provide information to assist the physician in navigating the medical instrument. Example suitable information may include real-time images captured by the medical instrument, a visualization to indicate an alignment or an orientation of the medical instrument relative to a target trajectory, a visualization to indicate a progress of inserting the medical instrument into the kidney towards the target location, or guidance on the percutaneous procedure, among other examples. Such information can be captured or obtained using various sensors disposed on or otherwise coupled to the robotic system.

As used herein, the term “case log” or “case data” refers to any runtime data that a medical system (including a robotic system) generates or collects during a medical procedure.

Case logs also may include any data that can be generated or otherwise collected by the medical system (including data input by a user) before the medical procedure (also referred to as “pre-operation data”) or after the medical procedure (also referred to as “post-operation data”). For example, case logs may include data representing the poses of the instruments of the medical system, poses of the robotic arms or manipulators of the medical system, user interface (UI) commands, system status, video data, and audio data, among other examples. However, raw case log data may not be suitable for evaluating the success of the medical procedure or performance of the physician, for providing intraoperative recommendations or insights, or for performing automated actions. For example, the raw case log data does not identify when needle insertion starts or how many stick attempts a user makes after selecting a target location for entering into the kidney. Existing case logs also do not provide information indicating what insertion site a physician has selected or a length between a patient's skin and a target within the calyx or papilla (also referred to as the “tract length”).

In some aspects, a medical system (or an analytics system associated therewith) may segment a medical procedure into various phases based on the actions performed during each of the phases and the timing of such actions. As a result, the medical system may analyze the case data in the context of the segmentation to generate phase-specific metrics (also referred to as “clinical metrics”), provide insights or recommendations, or perform automated actions, among other examples. For example, to compute a tract length, a system may first calculate a position of the needle tip at the time the user selects an insertion site on the skin and a target position of the calyx or papilla. However, such clinical metrics are only computable from the case logs after segmenting a percutaneous access medical procedure. Example phases of a PCNL access procedure may include, but are not limited to, a target selection phase (including any activities performed to set a target inside a patient's kidney), a site selection phase (from when a user selects the target to when the user positions the needle tip at a specific location on a patient's skin), a needle insertion phase (during which the user inserts the needle into the patient and stops with no intention to push the needle any further), and a post insertion phase (including any activities between consecutive needle insertion phases).

In some aspects, a medical system (or an inferencing system associated therewith) may analyze a set of clinical metrics associated with a medical procedure to determine relationships between the metrics, including any relationships to a successful performance of the medical procedure. Such relationships can be used to provide guidance or insights before, during, or after the medical procedure is performed. In some implementations, the medical system may determine statistical correlations between the various clinical metrics. For example, the medical system may determine that tract length is statistically correlated with the outcome of the medical procedure (such as whether the procedure is successful). While the statistical test for correlation can indicate whether two variables often occur together, it cannot explain whether one causes the other. For example, the medical system also may determine that the distance between the scope and a target (also referred to as the “target offset”) is statistically correlated with the steadiness of the needle during the insertion phase (based on a deviation of the needle tip relative to a principal insertion direction). In this example, a confounding variable (such as the tract length) may have a causal effect on both the target offset and the needle insertion steadiness. Thus, a statistical correlation between two variables is not sufficient, on its own, to expound a causal relationship between the variables.

Without knowing the causal relationships between various clinical metrics, a medical system can only offer limited inferences or insights about the medical procedure (or the medical system itself) based on the clinical metrics. For example, the medical system may identify several clinical metrics that are statistically correlated with a successful medical procedure, however, only a very small subset of the identified clinical metrics may have any causal effect on procedure success. As a result, a team that designs or manufactures the robotic system used in the medical procedure may invest a considerable amount of time and resources on investigating the root causes of customer complaints, or on improving the robotic system's software, hardware, or workflow based on the guidance of statistical relationships between clinical metrics (designed to measure the effectiveness of the software or hardware) that may have little or no causal effect on the success of the medical procedure. A physician or user of the medical system also may review each of the identified metrics to determine how they can improve the outcome. Without proper direction, the physician may focus their time and efforts on improving certain skills or techniques (such as decreasing wobble during needle insertion) or making better intra-operative decisions (such as choosing a different calyx for needle insertion) that have little or no causal effect on the success of the medical procedure. However, aspects of the present disclosure recognize that causal relationships can be extracted from data using various “causal discovery” techniques.

Various aspects relate generally to medical robotics, and more particularly, to determining causal relationships between clinical metrics associated with a medical procedure performed, at least in part, by a robotic system. In some aspects, an inferencing system may receive case data (also referred to as “telemetry data”) generated by a medical system configured to perform one or more operations associated with a medical procedure and determine a set of clinical metrics associated with the medical procedure based, at least in part, on the received case data. For example, the case data may indicate one or more poses, movements, or actuations of a robotic apparatus associated with the medical system, or one or more positions of an instrument or scope associated with the medical system. The inferencing system further includes a causal discovery component that maps the set of clinic metrics to a directed acyclic graph (DAG) based on one or more casual relationships between the various clinical metrics. For example, the DAG may indicate which of the clinical metrics are causally related, including which clinical metric has a causal effect on the other. In some implementations, the causal discovery component may perform the mapping based on one or more causal discovery algorithms. Example suitable causal discovery algorithms include the PC algorithm, greedy equivalence search (GES), additive noise models (ANM), and machine learning algorithms such as the NOTEARS algorithm, among other examples.

The inferencing system also includes an inferencing component configured to generate one or more inferences associated with the medical system based, at least in part, on the DAG. In some implementations, the inferences may include a visual representation of the DAG for display on an electronic display device. In some other implementations, the inferences may highlight or otherwise indicate one or more clinical metrics that caused a given instance of the medical procedure to fail. For example, the inferencing component may determine, based on the DAG and data distributions, the causal effect of each clinical metric on one another and on the success of the medical procedure. The inferencing component may further determine, for any set of clinical metrics having a causal relationship to the outcome of the procedure (also referred to as “causal metrics”), a range of values that result in a successful medical procedure. The inferencing component may compare each range of values to new values for the causal metrics (associated with the given instance of the medical procedure) to identify areas for improvement. In some other implementations, the inferences may highlight or otherwise indicate one or more clinical metrics that have a causal effect on a problem associated with a complaint investigation. For example, if the complaint indicates a problem with a given clinical metric, the inferencing component may determine, based on the DAG, which of the other clinical metrics have a causal effect on the given metric. Still further, in some implementations, the one or more inferences may include a simulated randomized controlled trial (RCT) using only observational data. For example, the inferencing component may use the DAG, identification, estimation, and refutation to predict the outcome of a medical procedure under varying conditions. The inferencing component also may use the DAG to perform counterfactual calculations.

Particular implementations of the subject matter described in this disclosure can be implemented to realize one or more of the following potential advantages. By identifying causal relationships between various clinical metrics, aspects of the present disclosure can provide greater insights about a robotically assisted medical procedure. For example, the DAG may indicate which clinical variable has a casual effect on the greatest number of other clinical variables or the greatest causal effect on the success of the medical procedure. Thus, research and development (R&D) teams can use the DAG to identify areas of the medical system that can be improved while yielding the maximum return on their investments. Physicians may use the inferences generated by the inferencing component to focus their time and effort on improving a narrow set of skills or techniques that may have the greatest impact on their performance of the medical procedure. Some inferences generated by the inferencing component also may be used to expedite or automate complaint investigations. For example, if a physician alleges that they were unable to access a kidney stone using the medical system, the inferencing component may identify a narrow set of clinic metrics that could have caused the problem. A technician could then review the case values for those causal metrics to diagnose the issue or query the inferencing system to produce counterfactual estimates. By simulating RCTs, aspects of the present disclosure may allow R&D teams to test new features for the medical system on real world data and make product claims without the expense of clinical trials.

Although certain aspects of the present disclosure are described in detail herein in the context of renal, urological, or nephrological procedures, such as kidney stone removal and treatment procedures, it should be understood that such context is provided for convenience and clarity, and the concepts disclosed herein are applicable to any suitable medical procedure. However, as mentioned, description of the renal or urinary anatomy and associated medical issues and procedures is presented herein to aid in the description of the concepts disclosed herein. In some implementations, the techniques and systems described herein are discussed in the context of a percutaneous procedure, which can include any procedure where access is gained to a target location by making a puncture or incision in the skin, mucous membrane, or other body layer. However, it should be understood that these techniques and systems can be implemented in the context of any medical procedure including, for example, minimally invasive procedures (such as laparoscopy), non-invasive procedures (such as endoscopy), therapeutic procedures, diagnostic procedures, percutaneous procedures, non-percutaneous procedures, or other types of procedures. Endoscopic procedures can include bronchoscopy, ureteroscopy, gastroscopy, nephroscopy, and nephrolithotomy, among other examples.

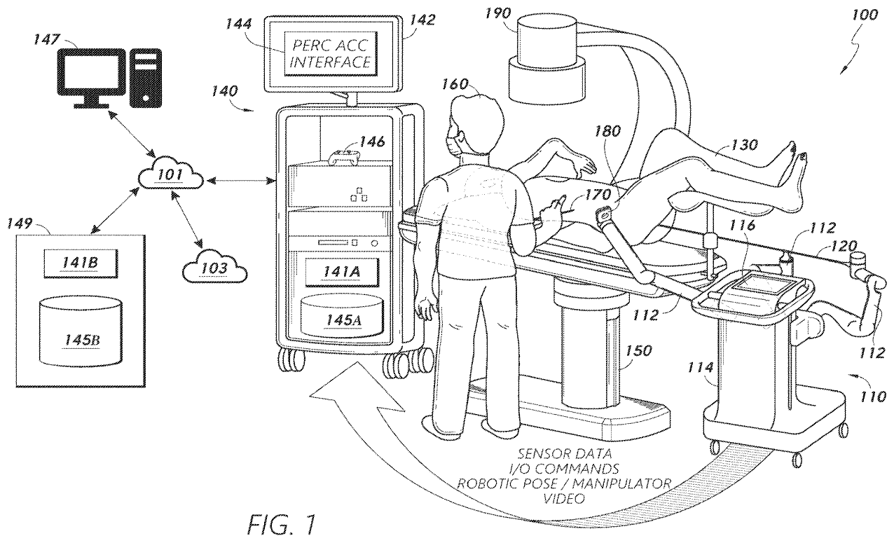

FIG. 1 shows an example medical system 100, according to some implementations. The medical system 100 includes a robotic system 110 configured to engage with or control a medical instrument to perform a procedure on a patient 130. The medical system 100 also includes a control system 140 configured to interface with the robotic system 110, provide information regarding the procedure, or perform a variety of other operations. For example, the control system 140 can include a display(s) 142 to present certain information to assist the physician 160. The display(s) 142 may be a monitor, screen, television, virtual reality hardware, augmented reality hardware, or three-dimensional imaging devices (such as hologram devices), among other examples, or combinations thereof. The medical system 100 can include a table 150 configured to hold the patient 130. The system 100 can further include an electromagnetic (EM) field generator 180, which can be held by one or more robotic arms 112 of the robotic system 110 or can be a stand-alone device. In examples, the medical system 100 can also include an imaging device 190 which can be integrated into a C-arm or configured to provide imaging during a procedure, such as for a fluoroscopy-type procedure. Although shown in FIG. 1, in some implementations the imaging device 190 is eliminated.

In some implementations, the medical system 100 can be used to perform a percutaneous procedure. For example, if the patient 130 has a kidney stone that is too large to be removed through a urinary tract, the physician 160 can perform a procedure to remove the kidney stone through a percutaneous access point on the patient 130. To illustrate, the physician 160 can interact with the control system 140 to control the robotic system 110 to advance and navigate the medical instrument (such as a scope) from the urethra, through the bladder, up the ureter, and into the kidney where the stone is located. The control system 140 can provide information via the display(s) 142 regarding the medical instrument to assist the physician 160 in navigating the medical instrument, such as real-time images captured therewith.

Once at the site of the kidney stone (such as within a calyx of the kidney), the medical instrument can be used to designate or tag a target location for the medical instrument (such as a needle) to access the kidney percutaneously (such as a desired point to access the kidney). To minimize damage to the kidney or the surrounding anatomy, the physician 160 can designate a particular papilla as the target location for entering into the kidney with the medical instrument. However, other target locations can be designated or determined. To assist the physician in driving the medical instrument into the patient 130 through the particular papilla, the control system 140 can provide a percutaneous access interface 144, which can include a visualization to indicate an alignment of an orientation of the medical instrument relative to a target trajectory (such as a desired access path from the patient's skin to the target location), a visualization to indicate a progress of inserting the medical instrument into the kidney towards the target location, guidance on the percutaneous procedure, or other information. Once the medical instrument has reached the target location (as determined, such as by sensors attached to the needle 170, the scope 120, or other any sensor or imaging modality), the physician 160 can use the medical instrument or another medical instrument to extract the kidney stone from the patient 130, such as through the percutaneous access point.

Although the above percutaneous procedure or other procedures are discussed in the context of using the medical instrument, in some implementations a percutaneous procedure can be performed without the assistance of the medical instrument. Further, the medical system 100 can be used to perform a variety of other procedures. Moreover, although many implementations describe the physician 160 using the medical instrument, the medical instrument can alternatively be used by a component of the medical system 100. For example, the medical instrument can be held or manipulated by the robotic system 110 (such as the one or more robotic arms 112) and the techniques discussed herein can be implemented to control the robotic system 110 to insert the medical instrument with the appropriate pose (or aspect of a pose, such as orientation or position) to reach a target location.

In the example of FIG. 1, one medical instrument is implemented as a scope 120 and another medical instrument 170 is implemented as a needle 170. Thus, for case of discussion, one medical instrument is referred to as “the scope 120” or “the lumen-based medical instrument,” and another medical instrument is referred to as “the needle 170” or “the percutaneous medical instrument.” However, the medical instruments can each be implemented as a suitable type of medical instrument including, for example, a scope (sometimes referred to as an “endoscope”), a needle, a catheter, a guidewire, a lithotripter, a basket retrieval device, forceps, a vacuum, a needle, a scalpel, an imaging probe, jaws, scissors, graspers, needle holder, micro dissector, staple applier, tacker, suction or irrigation tool, or clip applier, among other examples. In some implementations, a medical instrument is a steerable device, while some other implementations a medical instrument is a non-steerable device. In some implementations, a surgical tool refers to a device that is configured to puncture or to be inserted through the human anatomy, such as a needle, a scalpel, or a guidewire, among other examples. However, a surgical tool can refer to other types of medical instruments.

In some implementations, a medical instrument, such as the scope 120 or the needle 170, includes a sensor that is configured to generate sensor data, which can be sent to another device. In examples, sensor data can indicate a location or orientation of the medical instrument or can be used to determine a location or orientation of the medical instrument. For instance, a sensor can include an electromagnetic (EM) sensor with a coil of conductive material. Here, an EM field generator, such as the EM field generator 180, can provide an EM field that is detected by the EM sensor on the medical instrument. The magnetic field can induce small currents in coils of the EM sensor, which can be analyzed to determine a distance or angle or orientation between the EM sensor and the EM field generator. Further, a medical instrument can include other types of sensors configured to generate sensor data, such as one or more of any of: a camera, a range sensor, a radar device, a shape sensing fiber, an accelerometer, a gyroscope, a satellite-based positioning sensor (such as a global positioning system (GPS)), or a radio-frequency transceiver, among other examples. In some implementations, a sensor is positioned on a distal end of a medical instrument, while in some other implementations a sensor is positioned at another location on the medical instrument. In some implementations, a sensor on a medical instrument can provide sensor data to the control system 140 and the control system 140 can perform one or more localization techniques to determine or track a position or an orientation of a medical instrument.

In some implementations, the medical system 100 may record or otherwise track the runtime data that is generated during a medical procedure. For example, the medical system 100 may track or otherwise record the sensor readings (such as sensor data) from the instruments (such as the scope 120 and the needle 170) in case data store 145A (such as a computer storage system, such as computer readable memory, database, or filesystem, among other examples). In some implementations, the case data store 145A may include removable media (such as optical discs, flash memory, magnetic storage media, universal serial bus (USB) drives, and external solid state drives (SSDs) or hard disk drives (HDDs), among other examples) that can be used to transport the case logs to another computing system for analysis. In addition to sensor data, the medical system 100 can store other types of case logs in the case data store 145A. For example, in the context of FIG. 1, the case logs can include time series data of the video images captured by the scope 120, status of the robotic system 110, commanded data from an I/O device(s) (such as I/O device(s) 146), audio data (such as may be captured by audio capturing devices embedded in the medical system 100, such as microphones on the medical instruments, robotic arms, or elsewhere in the medical system), external (relative to the patient) imaging device (such as RGB cameras, LIDAR imaging sensors, or fluoroscope imaging sensors), among other examples.

As shown in FIG. 1, the control system 140 includes an analytics engine 141A which may operate on the case logs stored in the case data store 145A to label the case logs according to a procedure phase for a given time. In some implementations, the analytics engine 141A may employ machine learning techniques to segment the medical procedure according to the different phases. In some implementations, once the medical procedure has been segmented, the analytics engine 141A may generate metrics for the medical procedure phases, the medical procedure generally, or provide insights and recommendations to the users of the medical system 100 (such as physicians, staff, or training personnel).

FIG. 1 further shows that in some implementations the control system 140 may include a network connection (such as via network 101) to a cloud-based data analytics platform 149. The cloud-based data analytics platform 149 may be a computer system that provides third party computers 147 postoperative analytic capabilities on a given medical procedure or analytics across multiple medical procedures. As shown in FIG. 1, the cloud-based data analytics platform 149 may further connect to additional medical systems 103, which each in turn may transmit case logs to the cloud-based data analytics platform 149. Because the cloud-based data analytics platform receives case logs from multiple medical systems, the cloud-based data analytics platform 149 may have access to a comparatively larger pool of data than a single medical system would have access to and may in turn aggregate the case logs across multiple medical systems to derive medical procedure insights. Medical procedure insights may include guidance on factors that result in an increased likelihood for success in a medical procedure based on the metrics derived from segmenting the case logs across medical systems and across medical procedures.

As shown in FIG. 1, the cloud-based data analytics platform 149 may include an analytics engine 141B and cloud-based data store 145B. The cloud-based data store 145B may be a computer storage device that stores the system data records from the medical system 100 and the additional medical systems 103. The analytics engine 141B may include features and capabilities similar to the analytics engine 141A. However, in some implementations, the analytics engine 141A may further operate to analyze case logs across multiple medical systems (such as medical system 100 and medical systems 103) to generate metrics or insights. This may provide comparatively robust insights because the data used to generate such metrics or insights is using a broader range of information. Additionally, or alternatively, the cloud-based analytics engine 141B may uses machine learning techniques that are suitable for post-operative classification, whereas the local analytics engine 141B may use machine learning techniques that are suitable for real-time or near-real time classification.

The term “scope” or “endoscope” is used herein according to its broad and ordinary meanings and can refer to any type of elongate medical instrument having image generating, viewing, or capturing functionality and configured to be introduced into any type of organ, cavity, lumen, chamber, or space of a body. For example, references herein to scopes or endoscopes can refer to a ureteroscope (such as for accessing the urinary tract), a laparoscope, a nephroscope (such as for accessing the kidneys), a bronchoscope (such as for accessing an airway, such as the bronchus), a colonoscope (such as for accessing the colon), an arthroscope (such as for accessing a joint), a cystoscope (such as for accessing the bladder), or a borescope, among other examples.

A scope can comprise a tubular or flexible medical instrument that is configured to be inserted into the anatomy of a patient to capture images of the anatomy. In some implementations, a scope can accommodate wires or optical fibers to transfer signals to or from an optical assembly at a distal end of the scope, which can include an imaging device, such as an optical camera. The camera or imaging device can be used to capture images of an internal anatomical space, such as a target calyx or papilla of a kidney. In some implementations, the camera or imaging device may be disposed on or coupled to the distal end of the scope. In some other implementations, the camera or imaging device may be disposed on or coupled to a working channel that can be deployed via a lumen of the scope. A scope can further be configured to accommodate optical fibers to carry light from proximately-located light sources, such as light-emitting diodes, to the distal end of the scope. In some implementations, the light sources may be disposed on or coupled to the distal end of the scope. For example, the distal end of the scope can include ports for light sources to illuminate an anatomical space when using the camera or imaging device. In some other implementations, the light sources may be disposed on or coupled to a working channel that can be deployed via a lumen of the scope. In some implementations, the scope may be controlled by a robotic system, such as the robotic system 110. The imaging device can comprise an optical fiber, fiber array, or lens. The optical components can move along with the tip of the scope such that movement of the tip of the scope results in changes to the images captured by the imaging device.

A scope can be articulable, such as with respect to at least a distal portion of the scope, so that the scope can be steered within the human anatomy. In some implementations, a scope is configured to be articulated with, for example, five or six degrees of freedom, including X, Y, Z coordinate movement, as well as pitch, yaw, and roll. A position sensor(s) of the scope can likewise have similar degrees of freedom with respect to the position information they produce or provide. A scope can include telescoping parts, such as an inner leader portion and an outer sheath portion, which can be manipulated to telescopically extend the scope. A scope, in some instances, can comprise a rigid or flexible tube, and can be dimensioned to be passed within an outer sheath, catheter, introducer, or other lumen-type device, or can be used without such devices. In some implementations, a scope includes a working channel for deploying medical instruments (such as lithotripters, basketing devices, or forceps), irrigation, or aspiration to an operative region at a distal end of the scope.

The robotic system 110 can be configured to at least partly facilitate execution of a medical procedure. The robotic system 110 can be arranged in a variety of ways depending on the particular procedure. The robotic system 110 can include the one or more robotic arms 112 configured to engage with or control the scope 120 to perform a procedure. As shown, each robotic arm 112 can include multiple arm segments coupled to joints, which can provide multiple degrees of movement. In the example of FIG. 1, the robotic system 110 is positioned proximate to the patient's 130 legs and the robotic arms 112 are actuated to engage with and position the scope 120 for access into an access point, such as the urethra of the patient 130. When the robotic system 110 is properly positioned, the scope 120 can be inserted into the patient 130 robotically using the robotic arms 112, manually by the physician 160, or a combination thereof. The robotic arms 112 can also be connected to the EM field generator 180, which can be positioned near a treatment site, such as within proximity to the kidneys of the patient 130.

The robotic system 110 can also include a support structure 114 coupled to the one or more robotic arms 112. The support structure 114 can include control electronics or circuitry, one or more power sources, one or more pneumatics, one or more optical sources, one or more actuators (such as motors to move the one or more robotic arms 112), memory or data storage, or one or more communication interfaces. In some implementations, the support structure 114 includes an input/output (I/O) device(s) 116 configured to receive input, such as user input to control the robotic system 110, or provide output, such as a graphical user interface (GUI), information regarding the robotic system 110, or information regarding a procedure, among other examples. The I/O device(s) 116 can include a display, a touchscreen, a touchpad, a projector, a mouse, a keyboard, a microphone, or a speaker. In some implementations, the robotic system 110 is movable (such as the support structure 114 includes wheels) so that the robotic system 110 can be positioned in a location that is appropriate or desired for a procedure. In some other implementations, the robotic system 110 is a stationary system. Further, in some implementations, the robotic system 110 is integrated into the table 150.

The robotic system 110 can be coupled to any component of the medical system 100, such as the control system 140, the table 150, the EM field generator 180, the scope 120, or the needle 170. In some implementations, the robotic system is communicatively coupled to the control system 140. In one example, the robotic system 110 can be configured to receive a control signal from the control system 140 to perform an operation, such as to position a robotic arm 112 in a particular manner, or manipulate the scope 120, among other examples. In response, the robotic system 110 can control a component of the robotic system 110 to perform the operation. In another example, the robotic system 110 is configured to receive an image from the scope 120 depicting internal anatomy of the patient 130 or send the image to the control system 140, which can then be displayed on the display(s) 142. Furthermore, in some implementations, the robotic system 110 may be coupled to additional components of the medical system 100, such as a fluid management system (not shown for simplicity) that controls a delivery (also referred to as “irrigation”) or return (also referred to as “aspiration”) of fluids used for certain types of medical procedures.

The control system 140 can be configured to provide various functionality to assist in performing a medical procedure. In some implementations, the control system 140 can be coupled to the robotic system 110 and operate in cooperation with the robotic system 110 to perform a medical procedure on the patient 130. For example, the control system 140 can communicate with the robotic system 110 via a wireless or wired connection (such as to control the robotic system 110 or the scope 120, receive an image(s) captured by the scope 120), provide fluids to the robotic system 110 via one or more fluid channels, provide power to the robotic system 110 via one or more electrical connections, provide optics to the robotic system 110 via one or more optical fibers or other components, among other examples. Further, in some implementations, the control system 140 can communicate with the needle 170 or the scope 120 to receive sensor data from the needle 170 or the scope 120 (via the robotic system 110 or directly from the needle 170 or the scope 120). Moreover, in some implementations, the control system 140 can communicate with the table 150 to position the table 150 in a particular orientation or otherwise control the table 150. Further, in some implementations, the control system 140 can communicate with the EM field generator 180 to control generation of an EM field around the patient 130.

The control system 140 includes various I/O devices configured to assist the physician 160 or others in performing a medical procedure. In this example, the control system 140 includes an I/O device(s) 146 that is employed by the physician 160 or other user to control the scope 120, such as to navigate the scope 120 within the patient 130. For example, the physician 160 can provide input via the I/O device(s) 146 and, in response, the control system 140 can send control signals to the robotic system 110 to manipulate the scope 120. Although the I/O device(s) 146 is illustrated as a controller in the example of FIG. 1, the I/O device(s) 146 can be implemented as a variety of types of I/O devices, such as a touchscreen, a touch pad, a mouse, a keyboard, a surgeon or physician console, virtual reality hardware, augmented hardware, microphone, speakers, or haptic devices, among other examples.

As also shown in FIG. 1, the control system 140 can include the display(s) 142 to provide various information regarding a procedure. The display(s) 142 can present the percutaneous access interface 144 to assist the physician 160 in the percutaneous access procedure (such as manipulating the needle 170 towards a target site). The display(s) 142 can also provide (such as via the percutaneous access interface 144 or another interface) information regarding the scope 120. For example, the control system 140 can receive real-time images that are captured by the scope 120 and display the real-time images via the display(s) 142. Additionally, or alternatively, the control system 140 can receive signals (such as analog, digital, electrical, acoustic or sonic, pneumatic, tactile, hydraulic) from a medical monitor or a sensor associated with the patient 130, and the display(s) 142 can present information regarding the health or environment of the patient 130. Such information can include information that is displayed via a medical monitor including, for example, a heart rate (such as ECG or HRV), blood pressure or rate, muscle bio-signals (such as EMG), body temperature, blood oxygen saturation (such as SpO₂), CO₂, brainwaves (such as EEG), environmental or local or core body temperature, among other examples.

To facilitate the functionality of the control system 140, the control system 140 can include various components (sometimes referred to as “subsystems”). For example, the control system 140 can include control electronics or circuitry, as well as one or more power sources, pneumatics, optical sources, actuators, memory or data storage devices, or communication interfaces. In some implementations, the control system 140 includes control circuitry comprising a computer-based control system that is configured to store executable instructions, that when executed, cause various operations to be implemented. In some implementations, the control system 140 is movable, such as that shown in FIG. 1, while in some other implementations, the control system 140 is a stationary system. Although various functionality and components are discussed as being implemented by the control system 140, any of this functionality or components can be integrated into or performed by other systems or devices, such as the robotic system 110, the table 150, or the EM field generator 180 (or even the scope 120 or the needle 170).

The imaging device 190 can be configured to capture or generate one or more images of the patient 130 during a procedure, such as one or more x-ray or CT images. In examples, images from the imaging device 190 can be provided in real-time to view anatomy or medical instruments, such as the scope 120 or the needle 170, within the patient 130 to assist the physician 160 in performing a procedure. The imaging device 190 can be used to perform a fluoroscopy (such as with a contrast dye within the patient 130) or another type of imaging technique. Although shown in FIG. 1, in many implementations the imaging device 190 is not implemented for performing a procedure or the imaging device 190 (including the C-arm) is eliminated.

The various components of the medical system 100 can be communicatively coupled to each other over a network, which can include a wireless or wired network. Example networks include one or more personal area networks (PANs), local area networks (LANs), wide area networks (WANs), Internet area networks (IANs), cellular networks, or the Internet. Further, in some implementations, the components of the medical system 100 are connected for data communication, fluid or gas exchange, or power exchange, among other examples, via one or more support cables, or tubes, among other examples.

The medical system 100 can provide a variety of benefits, such as providing guidance to assist a physician in performing a procedure (such as instrument tracking or instrument alignment information), enabling a physician to perform a procedure from an ergonomic position without the need for awkward arm motions or positions, enabling a single physician to perform a procedure with one or more medical instruments, avoiding radiation exposure (such as associated with fluoroscopy techniques), enabling a procedure to be performed in a single-operative setting, or providing continuous suction to remove an object more efficiently (such as to remove a kidney stone), among other examples. For example, the medical system 100 can provide guidance information to assist a physician in using various medical instruments to access a target anatomical feature while minimizing bleeding or damage to anatomy (such as critical organs or blood vessels). Further, the medical system 100 can provide non-radiation-based navigational or localization techniques to reduce physician and patient exposure to radiation or reduce the amount of equipment in the operating room. Moreover, the medical system 100 can provide functionality that is distributed between at least the control system 140 and the robotic system 110, which can be independently movable. Such distribution of functionality or mobility can enable the control system 140 or the robotic system 110 to be placed at locations that are optimal for a particular medical procedure, which can maximize working area around the patient or provide an optimized location for a physician to perform a procedure.

Although various techniques and systems are discussed as being implemented as robotically-assisted procedures (such as procedures that at least partly use the medical system 100), the techniques and systems can be implemented in other procedures, such as in fully-robotic medical procedures, or human-only procedures (such as free of robotic systems), among other examples. For example, the medical system 100 can be used to perform a procedure without a physician holding or manipulating a medical instrument (such as a fully-robotic procedure). That is, medical instruments that are used during a procedure, such as the scope 120 and the needle 170, can each be held or controlled by components of the medical system 100, such as the robotic arm(s) 112 of the robotic system 110.

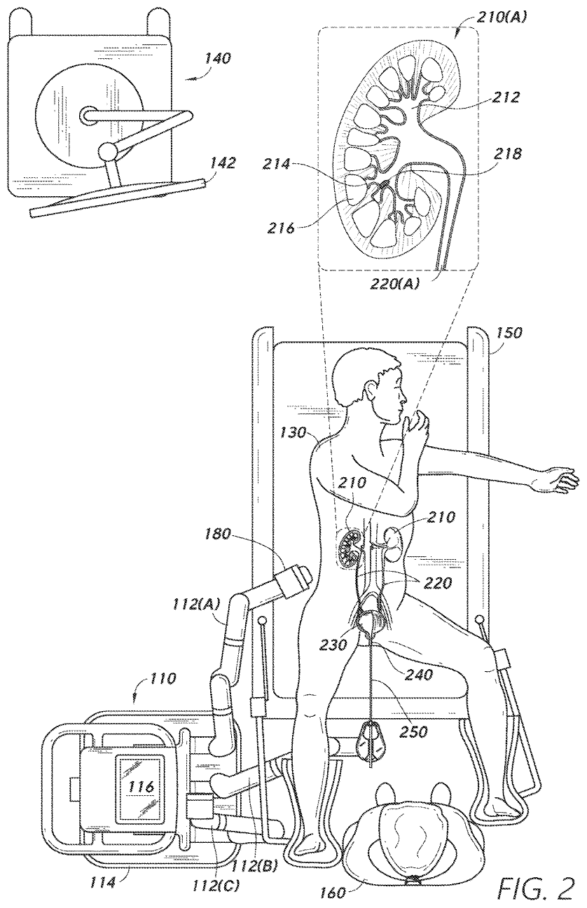

FIGS. 2-4 illustrate a top view of the medical system 100 of FIG. 1 arranged to perform a percutaneous procedure in accordance with one or more implementations. In these examples, the medical system 100 is arranged in an operating room to remove a kidney stone from the patient 130 with the assistance of the scope 120 and the needle 170. In some implementations of such a procedure, the patient 130 is positioned in a modified supine position with the patient 130 slightly tilted to the side to access the flank of the patient 130, such as that illustrated in FIG. 1. However, the patient 130 can be positioned in other manners, such as a supine position, or a prone position, among other examples. For ease of illustration in viewing the anatomy of the patient 130, FIG. 2-4 illustrate the patient 130 in a supine position with the legs spread apart. Also, for ease of illustration, the imaging device 190 (including the C-arm) has been removed.

Although FIGS. 2-4 illustrate use of the medical system 100 to perform a percutaneous procedure to remove a kidney stone from the patient 130, the medical system 100 can be used to remove a kidney stone in other manners or to perform other procedures. Further, the patient 130 can be arranged in other positions as desired for a procedure. Various acts are described in FIGS. 2-4 and throughout this disclosure as being performed by the physician 160. It should be understood that these acts can be performed directly by the physician 160, a user under direction of the physician, another user (such as a technician), a combination thereof, or any other user.

FIGS. 2-4 show various features of the anatomy of the patient 130. For example, the patient 130 includes kidneys 210 fluidly connected to a bladder 230 via ureters 220, and a urethra 240 fluidly connected to the bladder 230. As shown in the enlarged depiction of the kidney 210(A), the kidney 210(A) includes calyces (including calyx 212), renal papillae (including the renal papilla 214, also referred to as “the papilla 214”), and renal pyramids (including the renal pyramid 216). In these examples, a kidney stone 218 is located in proximity to the papilla 214. However, the kidney stone 218 can be located at other locations within the kidney 210(A) or elsewhere.

As shown in FIG. 2, to remove the kidney stone 218 in the example percutaneous procedure, the physician 160 can position the robotic system 110 at the side or foot of the table 150 to initiate delivery of the scope 120 (not illustrated in FIG. 2) into the patient 130. In particular, the robotic system 110 can be positioned at the side of the table 150 within proximity to the feet of the patient 130 and aligned for direct linear access to the urethra 240 of the patient 130. In examples, the hip of the patient 130 is used as a reference point to position the robotic system 110. Once positioned, one or more of the robotic arms 112, such as the robotic arms 112(B) and 112(C), can stretch outwards to reach in between the legs of the patient 130. For example, the robotic arm 112(B) can be controlled to extend and provide linear access to the urethra 240, as shown in FIG. 2. In this example, the physician 160 inserts a medical instrument 250 at least partially into the urethra 240 along this direct linear access path (sometimes referred to as “a virtual rail”). The medical instrument 250 can include a lumen-type device configured to receive the scope 120, thereby assisting in inserting the scope 120 into the anatomy of the patient 130. By aligning the robotic arm 112(B) to the urethra 240 of the patient 130 or using the medical instrument 250, friction or forces on the sensitive anatomy in the area can be reduced. Although the medical instrument 250 is illustrated in FIG. 2, in some implementations, the medical instrument 250 is not used (such as the scope 120 can be inserted directly into the urethra 240).

The physician 160 can also position the robotic arm 112(A) near a treatment site for the procedure. For example, the robotic arm 112(A) can be positioned within proximity to the incision site or the kidneys 210 of the patient 130. The robotic arm 112(A) can be connected to the EM field generator 180 to assist in tracking a location of the scope 120 or the needle 170 during the procedure. Although the robotic arm 112(A) is positioned relatively close to the patient 130, in some implementations the robotic arm 112(A) is positioned elsewhere or the EM field generator 180 is integrated into the table 150 (which can allow the robotic arm 112(A) to be in a docked position). In this example, at this point in the procedure, the robotic arm 112(C) remains in a docked position, as shown in FIG. 2. However, the robotic arm 112(C) can be used in some implementations to perform any of the functions of the robotic arms 112(A) or 112(C).

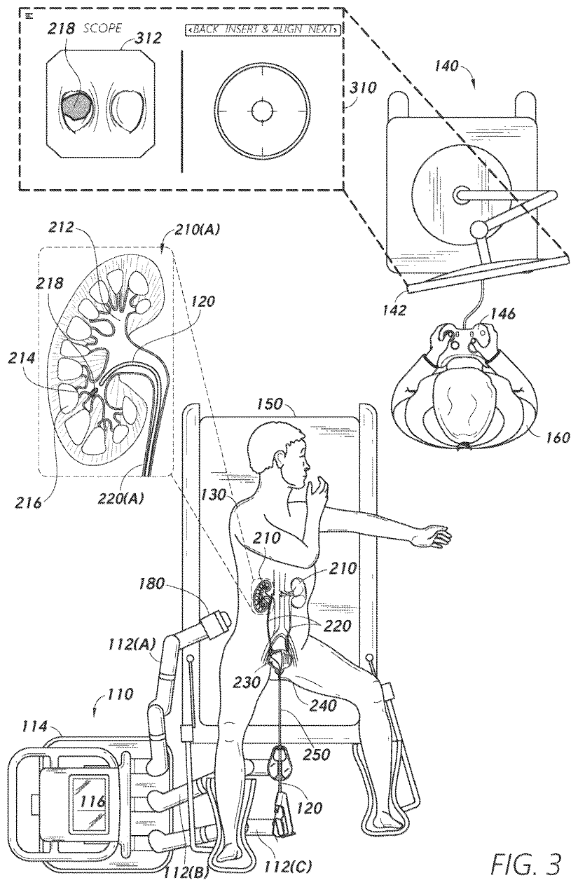

Once the robotic system 110 is properly positioned or the medical instrument 250 is inserted at least partially into the urethra 240, the scope 120 can be inserted into the patient 130 robotically, manually, or a combination thereof, as shown in FIG. 3. For example, the physician 160 can connect the scope 120 to the robotic arm 112(C) or position the scope 120 at least partially within the medical instrument 250 or the patient 130. The scope 120 can be connected to the robotic arm 112(C) at any time, such as before the procedure or during the procedure (such as after positioning the robotic system 110). The physician 160 can then interact with the control system 140, such as the I/O device(s) 146, to navigate the scope 120 within the patient 130. For example, the physician 160 can provide input via the I/O device(s) 146 to control the robotic arm 112(C) to navigate the scope 120 through the urethra 240, the bladder 230, the ureter 220(A), and up to the kidney 210(A).

As shown, the control system 140 can present an instrument-alignment interface 310, such as the instrument-alignment interface 310 of FIG. 3, via the display(s) 142 to view a real-time image 312 captured by the scope 120 to assist the physician 160 in controlling the scope 120. The physician 160 can navigate the scope 120 to locate the kidney stone 218, as depicted in the image 312. In some implementation, the control system 140 can use localization techniques to determine a position or an orientation of the scope 120, which can be viewed by the physician 160 through the display(s) 142 (not illustrated on the display(s) 142 in FIG. 3) to also assist in controlling the scope 120. Further, in some implementations, other types of information can be presented through the display(s) 142 to assist the physician 160 in controlling the scope 120, such as x-ray images of the internal anatomy of the patient 130.

Upon locating the kidney stone 218, the physician 160 can identify a location for the needle 170 to enter the kidney 210(A) for eventual extraction of the kidney stone 218. For example, to minimize bleeding or avoid hitting a blood vessel or other undesirable anatomy of the kidney 210(A) or anatomy surrounding the kidney 210(A), the physician 160 can seek to align the needle 170 with an axis of a calyx (such as can seek to reach the calyx head-on through the center of the calyx). To do so, the physician 160 can identify a papilla as a target location. In this example, the physician 160 uses the scope 120 to locate the papilla 214 that is near the kidney stone 218 and designate the papilla 214 as the target location. In some implementations of designating the papilla 214 as the target location, the physician can cause the medical system to tag the papilla. In tagging the papilla, the physician 160 can navigate the scope 120 to contact the papilla 214 and provide a UI input to the system to indicate the tagging, the control system 140 can use localization techniques to determine a location of the scope 120 (such as a location of the end of the scope 120), and the control system 140 can associate the location of the scope 120 with the target location. Additionally, or alternatively, the physician 160 can navigate the scope 120 to be within a particular distance to the papilla 214 (such as park in front of the papilla 214) and provide input indicating that the target location is within a field-of-view of the scope 120. The control system 140 can perform image analysis or other localization techniques to determine a location of the target location. In some other implementations, the scope 120 can deliver a fiduciary to mark the papilla 214 as the target location.

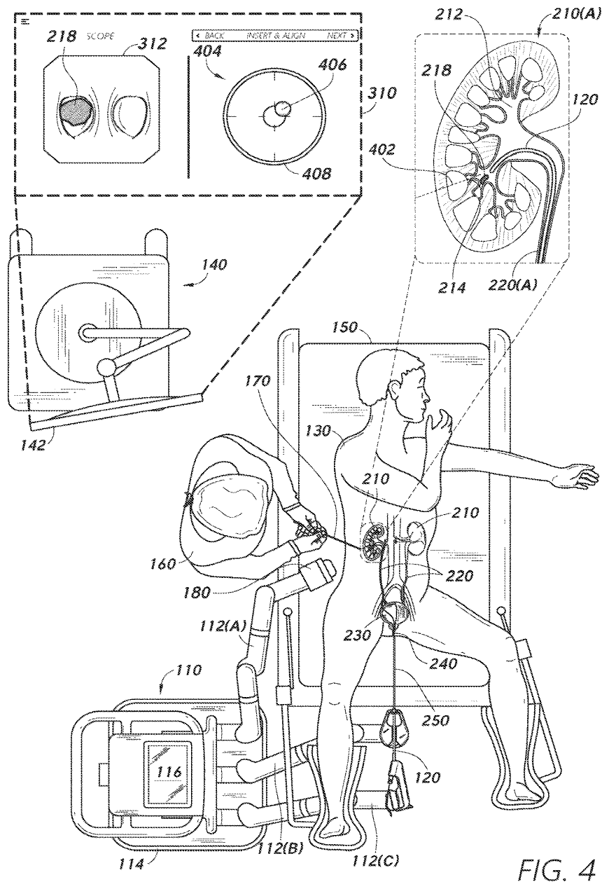

As shown in FIG. 4, the physician 160 can proceed with the procedure by positioning the needle 170 for insertion into the target location. In some implementations, the physician 160 can use his or her best judgment to place the needle 170 on the patient 130 at an incision site, such as based on knowledge regarding the anatomy of the patient 130, experience from previously performing the procedure, an analysis of CT or x-ray images or other pre-operative information of the patient 130, among other examples. Further, in some implementations, the control system 140 can provide information regarding a location to place the needle 170 on the patient 130. The physician 160 can attempt to avoid critical anatomy of the patient 130, such as the colon, paraspinal muscles, ribs, intercostal nerves, lungs, or pleura. In some examples, the control system 140 can use CT, x-ray, or ultrasound images to provide information regarding a location to place the needle 170 on the patient 130.

The control system 140 can determine a target trajectory 402 for inserting the needle 170 to assist the physician 160 in reaching the target location (such as the papilla 214). The target trajectory 402 can represent a desired path for accessing the target location. The target trajectory 402 can be determined based on a position of a medical instrument (such as the needle 170 or the scope 120), a target location within the human anatomy, a position or orientation of a patient, or the anatomy of the patient (such as the location of organs within the patient relative to the target location), among other examples. In this example, the target trajectory 402 includes a straight line that passes through the papilla 214 and the needle 170 (such as extends from a tip of the needle 170 through the papilla 214, such as a point on an axis of the papilla 214). However, the target trajectory 402 can take other forms, such as a curved line, or can be defined in other manners. In some examples, the needle 170 is implemented a flexible bevel-tip needle that is configured to curve as the needle 170 is inserted in a straight manner. Such needle can be used to steer around particular anatomy, such as the ribs or other anatomy. Here, the control system 140 can provide information to guide a user, such as to compensate for deviation in the needle trajectory or to maintain the user on the target trajectory.

Although the example of FIG. 4 illustrates the target trajectory 402 extending coaxially through the papilla 214, the target trajectory 402 can have another position, angle, or form. For example, a target trajectory can be implemented with a lower pole access point, such as through a papilla located below the kidney stone 218 shown in FIG. 4, with a non-coaxial angle through the papilla, which can be used to avoid the hip.

The control system 140 can use the target trajectory 402 to provide an alignment-progress visualization 404 via the instrument-alignment interface 310. For example, the alignment-progress visualization 404 can include an instrument alignment element 406 indicative of an orientation of the needle 170 relative to the target trajectory 402. The physician 160 can view the alignment-progress visualization 404 and orient the needle 170 to the appropriate orientation (such as the target trajectory 402). When aligned, the physician 160 can insert the needle 170 into the patient 130 to reach the target location. The alignment-progress visualization 404 can provide a progress visualization 408 (also referred to as “the progress bar 408”) indicative of a proximity of the needle 170 to the target location. As such, the instrument-alignment interface 310 can assist the physician 160 in aligning or inserting the needle 170 to reach the target location.

Once the target location has been reached with the needle 170, the physician 160 can insert another medical instrument, such as a power catheter, vacuum, or nephroscope into the path created by the needle 170 or over the needle 170. The physician 160 can use the other medical instrument or the scope 120 to fragment and remove pieces of the kidney stone 218 from the kidney 210(A).

In some implementations, a position of a medical instrument can be represented with a point, point set, or an orientation of the medical instrument can be represented as an angle or offset relative to an axis or plane. For example, a position of a medical instrument can be represented with a coordinate(s) of a point or point set within a coordinate system (such as one or more X, Y, Z coordinates) or an orientation of the medical instrument can be represented with an angle relative to an axis or plane for the coordinate system (such as angle with respect to the X-axis or plane, Y-axis or plane, or Z-axis or plane). Here, a change in orientation of the medical instrument can correspond to a change in an angle of the medical instrument relative to the axis or plane. Further, in some implementations, an orientation of a medical instrument is represented with yaw, pitch, or roll information. In some other implementations, an orientation of a medical instrument is represented in quaternion representation. Quaternion representation may avoid singularities present in a representation based on yaw, pitch, and roll.

In some implementations, a trajectory may refer to a pose. For example, a trajectory of a medical instrument can refer to a pose of the medical instrument, including or indicating both a position and orientation of the medical instrument. Similarly, a target trajectory can refer to a target pose, including or indicating both a position and orientation of a desired path. However, in some other implementations, a trajectory refers to either an orientation or a position.

Although particular robotic arms of the robotic system 110 are illustrated (or described herein) as performing particular functions in the context of FIGS. 2-4, any of the robotic arms 112 can be used to perform the functions. Further, any additional robotic arms or systems can be used to perform the procedure. Moreover, the robotic system 110 can be used to perform other parts of the procedure. For example, the robotic system 110 can be controlled to align or insert the needle into the patient 130. To illustrate, one of the robotic arms 112 can engage with or control the needle 170 to position the needle 170 at the appropriate location, align the needle 170 with the target trajectory, or insert the needle 170 to the target location. The control system 140 can use localization techniques to perform such processing. As such, in some implementations, a percutaneous procedure can be performed entirely or partially with the medical system 100 (such as with or without the assistance of the physician 160).

As described with reference to FIGS. 2-4, a percutaneous medical procedure may have a number of phases. To simplify the discussion of implementations contemplated by this disclosure, four example phases are now provided. However, it should be appreciated that some other implementations can have more, less, or even different phases and the following phases are provided merely as an illustration and not a limitation.

Target selection: This phase can include the activities performed to set a target inside the kidney. For example, as described above, a ureteroscope can be driven to a target calyx, tag a papilla, parked at a distance away from the papilla, and “Set” to set a live target.

Site selection: This phase can include the activities from when the user selects a target to the moment when the user positions the needle tip at a specific location on the patient's skin with the intention to puncture the skin towards the kidney.

Needle Insertion: This phase includes time periods during which a user inserts the needle into the patient and stops with no intention to push the needle further. During needle insertion, a user can puncture the skin and insert the needle from the skin towards the target. This attempt may be referred to as a first needle insertion. If the user misses the target, a medical system may instruct the user to retract the needle (in some implementations, while keeping the needle inside the body) and attempt another needle insertion (such as a secondary needle insertion). Even though there is a distinction between first needle insertion and the secondary insertion attempts, some implementations may treat them as the same while some other implementations may treat them as different attempts.

Post Insertion: This phase encompasses the activities between consecutive or sequential needle insertion phases, if such exist, and the duration from the last needle insertion until the end of the percutaneous procedure.

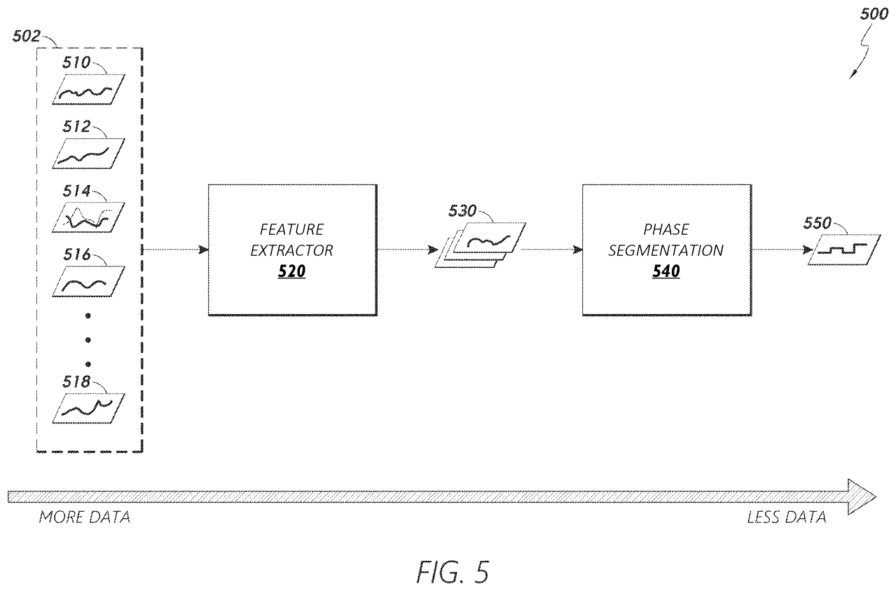

FIG. 5 shows a block diagram of an example analytics engine 500, according to some implementations. As described with reference to FIG. 1, the analytics engine 500 may receive and process the case logs 502 intraoperatively or postoperatively. In implementations that receive and process the case logs intraoperatively, the analytics engine 500 may be a component of a control system of a medical system, such as the analytics engine 141A of the control system 140 shown in FIG. 1. In implementations that receive and process the case logs postoperatively, the analytics engine 500 may be a component of a cloud-based data analytics platform connected to a medical system (or medical systems), such as the analytics engine 141B of the cloud-based data analytics platform 149 shown in FIG. 1.

As FIG. 5 shows, the analytics engine 500 may receive case logs 502. As previously discussed, case logs may include runtime data a medical system collects and otherwise stores during the operation of the medical system as a medical procedure is performed. As FIG. 5 shows, the case logs 502 may include robotic telemetry data 510, instrument telemetry data 512, imaging data 514, device status data 516, and I/O data 518. The robotic telemetry data 510 may be time series data that represents the various poses, movements, and actuations of the robotic system, including the robotic arm joints and actuators. Such poses, movements, actuations of the robotic system may be part of a kinematics model of the system that can estimate not only the pose of the robotic arms but also, with known properties of the instrument, the pose of the instrument attached and otherwise controlled by the robotic system, such as a robotically controlled endoscope.

The instrument telemetry data 512 includes time series data that represents the positioning of the instruments based on sensor data. For example, a percutaneous needle may include an EM sensor that allows the system to record and determine the needle's location within an EM space. The system may record this EM space data to track the needle's location over time. In some implementations, the instrument telemetry data 512 may include sensor data other than EM data, such as shape sensing fiber, accelerometers, a gyroscope, a satellite-based positioning sensor (such as a global positioning system (GPS)), or a radio-frequency transceiver, among other examples, embedded on a medical instrument.

The imaging data 514 may be data that represents images or video captured by the camera on the end of the endoscope instrument. For example, a video may represent a sequence of images (also referred to as “frames”) that are displayed or captured in succession. In some implementations, the operating room or the robotic system may be equipped with imaging devices external to the patient. In such implementations, the imaging data 514 may include data received from such external imaging devices.

The device status data 516 may include time series data that represent, among other things, statuses of components of the medical system, statuses of the instruments connected to the medical system, the user interface menu page shown to the users by the medical system, and any other suitable device status. As an example, the medical system 100 may include radio-frequency identification (“RFID”) readers capable of reading RFID tags on medical instruments attached to the robotic system. The device status data 516 may include indicators associated with the medical instruments, as obtained by those RFID tags. Still further, in some implementations, where the medical instruments include EM sensors, the device status data 516 may include data indicative of whether the medical instrument is connected to the EM system and whether the medical instrument is within an EM field of an EM field generator of the EM system. The device status data 516 may further include a state of the robot. For example, the robot could be in scope driving state, basketing state, or lasering state, among other examples. This robot state is derived from instrument connection status and what UI page is being displayed to the user (which may, for example, be identified in the data of the I/O data 518).

The I/O data 518 may include time series data that represent, among other things, the commands transmitted by the I/O devices of the medical system. For example, the I/O data 518 may include data representing instrument drive commands (such as insert or retract) or articulation commands (such as articulate left, articulate right, articulate up, articulate down, roll scope). In some implementations, the I/O data 518 may include additional I/O commands that may represent events in the percutaneous procedures, like tag a papilla and parking the scope. These events may be determined based on the UI page currently being displayed by the medical device system. The I/O data 518 may, additionally or alternatively, include data representing the user's interactions with the UI displayed by the percutaneous access interface 144.

As used herein, “timeseries data” may include measurements or events that are tracked, monitored, down sampled, or aggregated over time. In some cases, the timeseries data may be a stream of data from a sensor according to some determinable frequency (such as based on sample rate of a sensor or event logger). In those cases, time may be derived based on some known time and the position of the sensor data in the stream. In other cases, the timeseries data may include events generated from the system. In those cases, the events may include a timestamp (or a range of timestamps) that identifies the time in which the event occurred and possible duration. In some implementations, the duration of an event may be inferred based on the occurrence of the next timestamp event. In other cases, events may be represented as a stream of events currently triggered in the stream, and timing may be inferred from a known time and the position of the event in the stream of events.

FIG. 5 shows that a feature extractor module 520 receives the case logs 502. The feature extractor module 520 is a module that analyzes the case logs 502 and outputs a set of one or more case log features 530. Case log features can include features related to the scope position in a world frame (such as the coordinate frame of the robotic system), the needle position in a world frame, target position in the world frame, distances between components (such as needle, scope, or target), or orientation between components, among other examples.

A phase segmentation module 540 receives the case log features 530 and generates a times series class prediction for whether the medical procedure is within a determinable phase, shown as segmented procedure data 550. In the case of percutaneous access, the phase segmentation module 540 may output a binary classification where class A represents ‘Needle Insertion Phase’ and class B represents ‘Other Phase’. In some other implementations, the phase segmentation module 540 may output a multi-class classification where class A represents ‘Needle Insertion Phase’, class B represents ‘Target Selection Phase’, class C represents ‘Site Selection Phase’, and class D represents ‘Post-Needle Insertion Phase’. These classifications are merely examples and some implementations may have more, less, or different classifications depending on the medical procedure.

The phase segmentation module 540 may estimate a phase of the medical procedure based on any suitable machine learning method, including but not limited to, hidden Markov models (HMM), long-term-short-memory networks, and deep-learning based methods, such as convoluted neural networks (“CNNs” or “CNN” in the singular). In some cases, additionally or alternatively, an engineered approach to phase estimating may be used with the machine learning methods. For example, an engineered approach may use conditional logic to determine whether the medical procedure is in a ‘Needle Insertion Phase.’

As FIG. 5 shows, the data processed by the system may reduce in terms of data size as data moves from left to right. For example, the case logs 502 are comparatively larger in data volume than the extracted feature data of the case log features 530 because the feature extractor module 520 narrows down the data from the case logs into the features that are impactful for phase segmentation. To accomplish this, the feature extractor module may combine multiple data values from the case logs to produce one value or may selectively filter out data that is not useful in the segmentation algorithms. Further reducing the data size, the extracted feature data of the case log features 530 is comparatively larger in data volume than the segmented procedure data 550, as the segmented procedure data 550 reduces the potentially many features over time into a classification over time. By reducing the dataset and ensuring that the data is relevant and suited to the segmentation algorithms, the systems and methods described herein offer a segmentation solution that is highly computationally efficient. Further, while this disclosure is largely directed to percutaneous medical procedures, it has greater clinical utility and can be adapted to a variety of procedures. Accordingly, data can be filtered with respect to a respective medical procedure of interest, and one can avoid the computational overhead involved in looking at the entire data universe of the medical procedure.

As described with reference to FIG. 5, the phase segmentation module 540 may receive a number of case log features to segment the medical procedure based on phases. By way of example and not limitation, exemplary case log features that a phase segmentation module may receive include: a position, orientation, or heading of a medical instrument; a position or orientation of a target; distances between various medical instruments or objects (such as a target); and a (linear or angular) speed or velocity of a medical instrument.

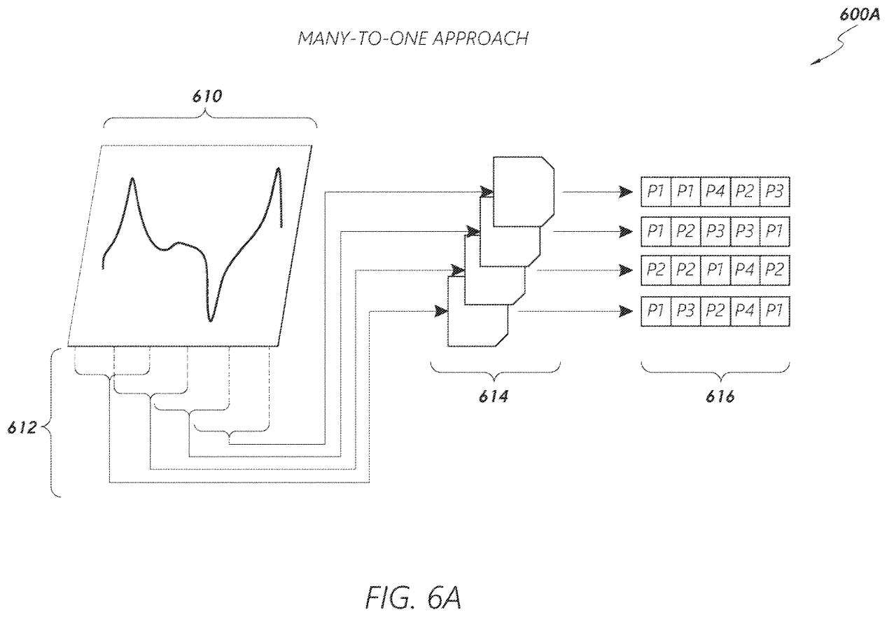

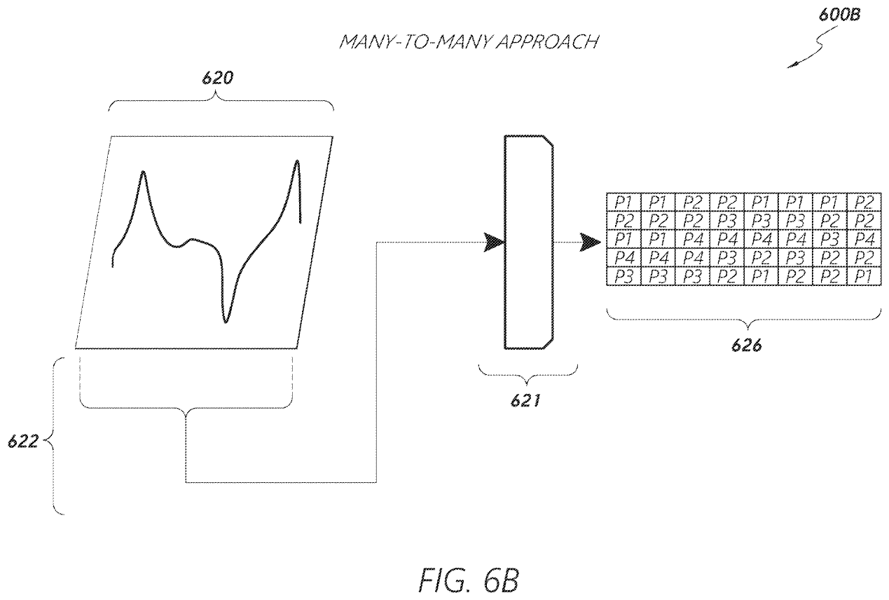

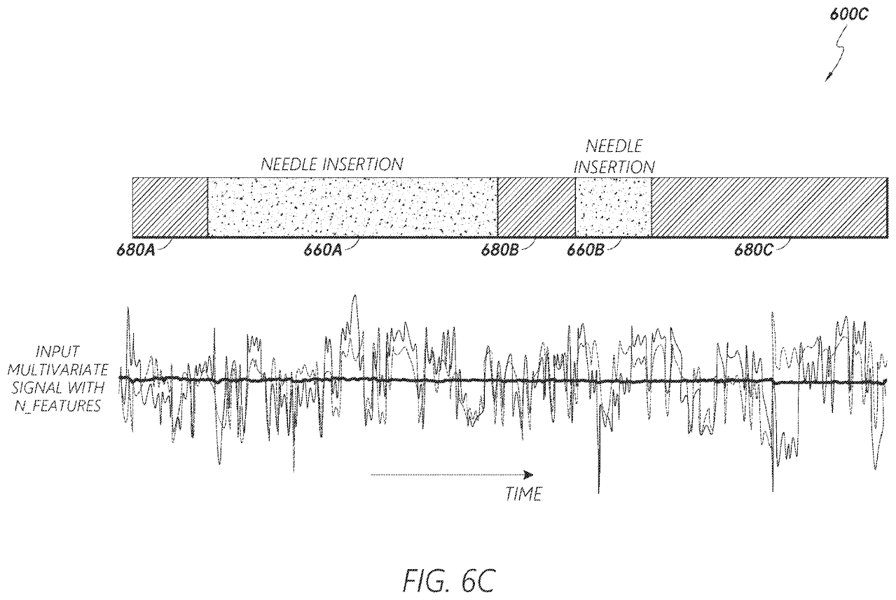

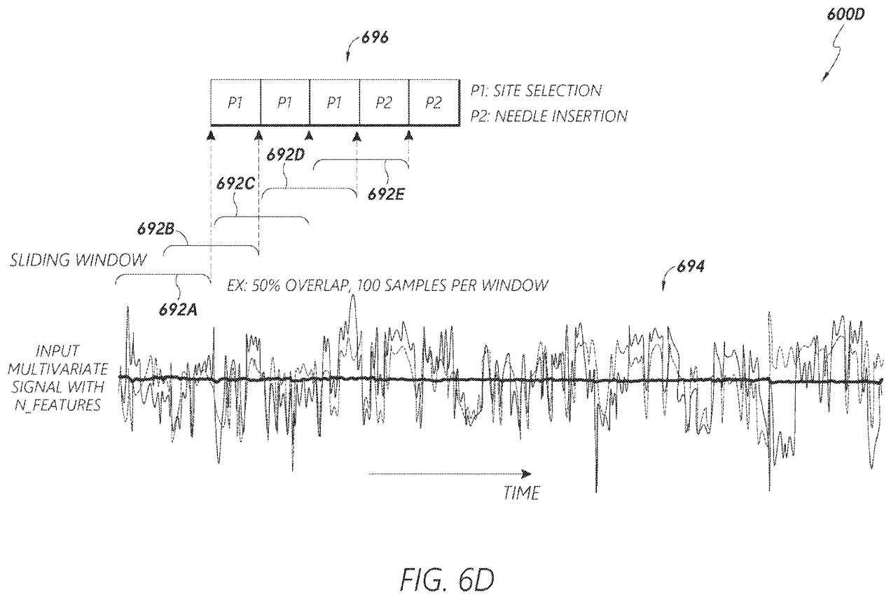

Aspects of the present disclosure may segment a medical procedure according to procedure phases using data (such as features) derived from case logs. Segmentation in this context may be solved as a classification problem where the time series data (such as data derived from the case logs) are classified with corresponding medical procedure phases to which the time series data belongs. Various aspects of classification are now discussed with reference to FIGS. 6A-D.

FIG. 6A shows a block diagram of an example many-to-one phase segmentation module 600A, according to some implementations. As FIG. 6A shows, the many-to-one phase segmentation module 600A may receive input time series 610. The input time series 610 may be case log features. Case log features are discussed in greater detail with reference to FIG. 5, but, without limitation, may include data derived from robotic telemetry data, instrument telemetry data, video data, device status data, or I/O data, among other examples. As shown in FIG. 6A, the many-to-one phase segmentation module 600A may divide the input time series 610 data into windowed segments 612, which are then input to the model evaluation block 614. In the model evaluation block 614, there are one or a plurality of model evaluations, one for each windowed segment. Each model evaluation then generates output predictions for the corresponding phase segments.

The many-to-one phase segmentation module 600A can provide phase segmentation for comparatively smaller window feature segments and may not necessarily require a complete set of data characterizing the medical procedure. As such, a many-to-one phase segmentation approach, as shown in FIG. 6A, may be used for real-time or near real-time phase detection (such as intraoperative phase detection). Further, given the intraoperative applicability, a local analytics engine (such as the local analytics engine 141A in FIG. 1) may utilize a many-to-one segmentation module similar to the many-to-one phase segmentation module 600A shown in FIG. 6A.

In some implementations, a many-to-one phase segmentation may be near real-time but not real-time because such an approach is delayed by the window feature segment. One approach that can be used by some other implementations to make many-to-one phase segmentation approaches real-time is to use a time series prediction module. Time series prediction takes in a window feature segment and predicts the next window feature segment. A time series prediction module can include a time series prediction network trained to predict the next window feature segment. During execution of the many-to-one phase segmentation, features would feed into a time series prediction network to predict the next window of features. The output of the time series prediction network would then feed into the time series classification of the many-to-one phase segmentation to predict the phase of the procedure for that window feature segment.

FIG. 6B shows a block diagram of an example many-to-many phase segmentation module 600B, according to some implementations. As FIG. 6B shows, the many-to-many phase segmentation module 600B may receive time series data 620. The time series data 620 may be features derived from case logs. Case log features are described with reference to FIG. 5 but may include, without limitation, data derived from robotic telemetry data, instrument telemetry data, video data, device status data, or I/O data, among other examples. As shown in FIG. 6B, the many-to-many phase segmentation module 600B may divide the input timeseries 620 into comparatively longer windowed feature segments 622 with respect to the many-to-one phase segmentation module 600A shown in FIG. 6A, which are then input to the model evaluation block 621. In the model evaluation block 621, there are few (or comparatively fewer) model evaluations, and many (or comparatively more) output predictions 626 for the corresponding feature segments.

The many-to-many phase segmentation module 600B may operate with a single model evaluation and can be applied to time series data of arbitrary length. As such, the many-to-many phase segmentation module 600B may be useful for post-operative analytics given that many-to-many phase segmentation module 600B may be more robust compared to the many-to-one phase segmentation module 600A. When used post-operatively, the training set used by the many-to-many phase segmentation module 600B may include data obtained from multiple prior procedures, potentially via multiple medical systems, and this training set may be built up over time. To illustrate with temporary reference to FIG. 1, the cloud-based data analytics platform 149 may utilize a many-to-many segmentation module similar to the many-to-many phase segmentation module 600B shown in FIG. 6B. The model evaluation used by the cloud-based data analytics platform 149 may be trained using data obtained from (or derived from data obtained from) the additional medical systems 103.

FIG. 6C shows a timing diagram depicting an example binary classification that can be performed by a segmentation module, according to some implementations. As shown, based on the time series data derived from the case log features, a segmentation module may generate medical procedure phase classifications in the form of a time series labels. The medical procedure phase classifications may be binary classifications that identify whether the predicted phase is the Needle Insertion phase (such as classifications 660A and 660B) or not (such as classifications 680A, 680B, 680C). In some implementations, the segmentation module may then execute higher-level decision processing to uncover the other classifications within the not ‘Needle Insertion’ phase (such as classifications 680A, 680B, 680C). This approach may be useful in a fully convolutional neural network, such as U-Net.

Rather than using binary classification, some implementations of a segmentation module (such as many-to-one phase segmentation module 600A or many-to-many phase segmentation module 600B) may use a multi-class classification architecture. For example, rather than merely classifying the medical procedure phase as Needle Insertion or Not, implementations may further classify the time series data to further include one or more of: Target Selection, Site Selection, or Post Needle Insertion, among other examples.

FIG. 6D shows an example output 600D of a segmentation module, according to some implementations. Further the output 600D is shown in the context of a many-to-many classification, where there are sliding segmentation windows 692A-E. Here, each of the sliding segmentation windows 692A-E correspond to different time periods in the segmentation features 694. Further, a sliding segmentation window may overlap in time with neighboring sliding segmentation windows. For example, sliding segmentation window 692B may correspond to a time period that overlaps the time period associated with sliding segmentation window 692A and a time period that overlaps with the time period associated with sliding segmentation window 692C. In some cases, there is a 50% overlap but this overlap may be different depending on the application and the size of the data sets used to train the models. For example, other overlaps may include overlaps of 25% or less given enough training data. The window lengths are also application specific, and this disclosure does not limit itself to any particular window length.

The sliding segmentation windows 692A-E are then used (such as by one or more evaluations of model evaluation block 614) to generate the medical procedure phase classifications 616.

FIG. 6D may represent an approach used by a parallel CNN long short-term memory (“LSTM”). A Parallel CNN-LSTM may use CNN layers and an attention LSTM layer in parallel. This contrasts with common serial CNN LSTM architectures, where CNNs are used to encode features and the LSTM layer is used to model long-term dependencies of the features across time. A Parallel CNN-LSTM can be used in a many-to-one architecture, as described with reference to FIG. 6A.

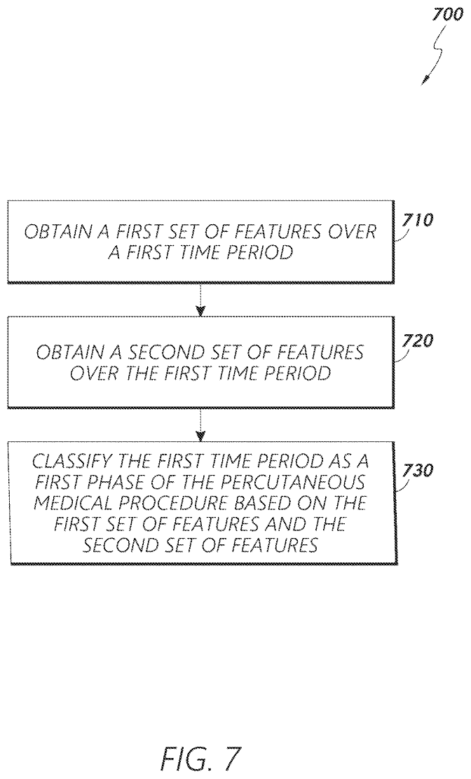

FIG. 7 shows an illustrative flowchart depicting an example operation 700 for evaluating phases in a percutaneous medical procedure, according to some implementations. For case in describing the operation 700, the operations of the operation 700 are described relative to the analytics engine 141A shown in FIG. 1. However, in actual implementations, the operation 700 may be performed by any of the components, alone or in combination, discussed herein.

At step 710, the analytics engine 141A may obtain a first set of features over a first time period. The first set of features may be derived at least partially from instrument telemetry data corresponding to an endoluminal scope (such as scope 120). As described with reference to FIGS. 1-4, the scope instrument may be a robotically controlled instrument entering the patient's body through an endoluminal entrance, such as the urinary tract through the urethra. The scope telemetry features may include data derived from sensors attached to the scope (such as a location sensor, such as an EM sensor, or shape sensor fiber, among other examples) that characterizes one or more of: the position, orientation, movement of the scope instrument over time (such as the first time period). For example, a feature extractor module may derive the scope instrument telemetry features from case logs generated by the medical system over the course of performing the medical procedure. The scope telemetry features may include scope position, scope orientation, relative needle scope heading, relative needle scope 3d distance, alignment error, scope linear body velocity, scope angular body velocity, or scope absolute speed, among other examples.

At step 720, the analytics engine 141A may obtain a second set of features over the first time period, the second set of features being derived at least in part from a percutaneous needle instrument. As described with reference to FIGS. 1-4, the needle instrument may be an instrument entering the patient's body percutaneously, such as through the skin of the flank of the patient. The needle telemetry features may be data derived from sensors attached to the needle (such as a location sensor, such as an EM sensor, or shape sensor fiber, among other examples) that characterizes one or more of: the position, orientation, movement of the needle instrument over time (such as the first time period). For example, a feature extractor module may derive features related to the telemetry of the needle instrument from case logs generated by the medical system over the course of performing the medical procedure. The features related to the needle instrument telemetry may include needle position, needle orientation, relative needle scope heading, relative needle scope 3d distance, distance to target, distance to target plane, alignment error, needle linear body velocity, needle angular body velocity, or needle absolute speed, among other examples.

At step 730, the analytics engine 141A may classify at least a portion of the first time period as a first phase of the percutaneous medical procedure based on the first set of features and the second set of features. For example, the percutaneous medical procedure can, depending on implementation, include at least the following phases: needle insertion, target selection, site selection, and post needle insertion. In some implementations, the classifier may generate classifications for multiple phases. In some other implementations, the classifier may generate binary classifications, such as needle insertion or not, where the Not classification signifies that the class may be any class other than a specified class. In some implementations that generate binary classifications, the system may perform high-level decision mechanisms to reclassify the time periods associated with the Not classification, to uncover the phases of those time periods. Classification of time period may include identifying the start and end time of the classified phase. This may be accomplished by including a timestamp representing a time in which the phase begins. To identify the end of the classified phase, implementations may include a duration associated with the classified phase or a timestamp representing a time in which the phase ends. Still further, some implementations may represent the end of a phase based on an inference, such as the beginning of the next phase. In this inference approach, an implementation may classify Phase1 as starting at Time1 and Phase2 starting at Time2. Embodiments may then infer that Phase1 ends when Phase2 begins (such as Time2).

Although not shown, the operation 700 may have additional steps. For example, in some implementations, steps 710, 720, and 730 could be performed with respect to additional time periods. In doing so, the operation 700 obtains features from a different time period and then classifies at least a portion of that time period with another (potentially different) classification corresponding to a phase of the percutaneous medical procedure. This additional classification can be performed according to a many-to-one approach or a many-to-many approach. FIGS. 6C and 6D illustrate classification of the time period 600C and time period 600D associated with features derived from the telemetry data. However, classification can use other case log data other than telemetry data and does not necessarily operate sequentially. Instead, some or all of the operations may be performed in parallel or otherwise overlapping. For example, in a many-to-many approach, all the phase classifications could be output at the same time based on case log data of the entire (or a significant portion of the) medical procedure.

In another example of an additional operation for the operation 700 not shown in FIG. 7, an implementation may obtain case log features related to a target, wherein the step 730 further classifies the first time period with the classification based additionally on the case log features related to the target.

Classifying the data from the case logs according to a phase in the percutaneous procedure may have a number of benefits. Such is the case because the information available to users from just the case logs may be limited to: the poses of instruments when field generator is in use, robot pose, and the pendant commands. Yet this information does not directly identify when needle insertion starts or how many attempts a user makes after selecting a target. The case logs also do not have direct data on what insertion site the user has selected. For example, to be able to compute the tract length, the anatomical position of the selected insertion sites, the system needs to know the pose of the needle, the position of the target, and “when” needle insertion starts. Embodiments discussed herein are capable of identifying what activity is going on in a procedure at a given time, so that the system can compute clinical metrics. Such metrics may be useable data for the physicians, as intraoperative data or as postoperative assessments. Furthermore, in some cases, data analytics on the metrics obtained over many medical procedures may uncover important factors that have impact on the success of a percutaneous procedure.

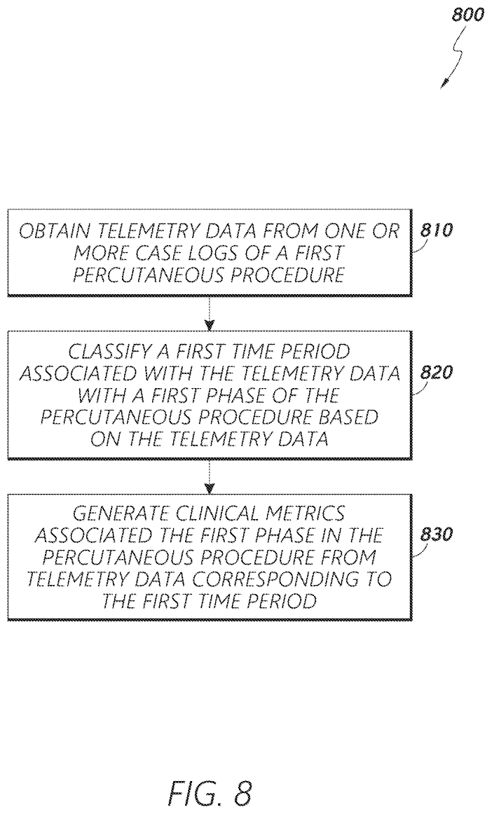

FIG. 8 shows an illustrative flowchart depicting an example operation 800 for generating clinical metrics associated with a percutaneous medical procedure, according to some implementations. For ease in describing the operation 800, the operations of the operation 800 are described relative to the analytics engine 141A shown in FIG. 1. However, in actual implementations, the operation 800 may be performed by any of the components, alone or in combination, discussed herein.

The analytics engine 141A obtains, at step 810, telemetry data from one or more case logs of a first percutaneous procedure. As discussed, the telemetry data may characterize the sensor readings of a needle instrument over time. In some implementations, at step 810, the analytics engine may receive additional data, such as robotic telemetry data, instrument telemetry data (such as sensor data), video data, device status data, and pendant command data.

At step 820, the analytics engine 141A classifies a first time period associated with the telemetry data with a first phase of the percutaneous procedure based on the telemetry data. The classification performed in step 820 may be performed using any of the classification methods discussed herein, such as using a machine learning approach that extracts features from the case log (or case logs) and evaluates them using a segmentation module, as shown in FIG. 5. Thus, although step 820 is discussed as only classifying at least a portion of the first time period, aspects of the present disclosure contemplate implementations that may classify multiple time periods. And further, such implementations may classify the first time period or any other time period based on case log data corresponding to time periods outside of the first time period. For example, where a many-to-many approach is utilized, case log data covering the entire medical procedure may be used and estimates of the phases may be output at the same time.

At step 830, the analytics engine 141, responsive to the first time period being associated with the first phase, generates clinical metrics associated the first phase in the percutaneous procedure from telemetry data corresponding to the first time period.

The types of metrics that can be generated by the analytics engine are now described. In some aspects, these metrics may be generated for an individual procedure or can provide global insight by collecting data from many procedures and then generating historical averages or correlated metrics with successful procedures.

Target Selection Phase: number of targeted calyces, kidney survey time, pole, retraction distance (distance the scope retracts after tagging the papilla), target selection duration, target offset (distance between a tip of the scope and an EM-target that the user created intra-operatively), target angle (difference in angle between the position of the scope when tagging a target location and the position of the scope at park position), anterior vs posterior calyx, parked pose, and respiration (represented by a deviation of a tip of the scope relative to a direction of movement of the scope).

Site Selection Phase: site selection time, average tract length, anatomical angles (cranial-caudal angle or anterior-posterior angle of the scope), distance of one or more instruments (such as a scope or needle) with respect to a field generator, coaxiality (angle between the needle instrument and the scope), tract length (distance between a tip of the instrument and a desired location), and angle to calyx (angle between a measured needle trajectory and the orientation or heading of the calyx).

Needle Insertion Phase: success (binary indication of whether the physician gained access to the kidney, by visualizing the cannula or trocar), needle insertion accuracy (as may be measured by a positional or angular deviation of the needle trajectory from a principal direction), needle insertion steadiness or stability (deviation of a tip of the instrument relative to a principal insertion direction), shifts in scope position or heading, accuracy as measured by a UI element displayed to user, number of attempts, needle insertion or retraction speed or acceleration, distance to target (shortest distance between a tip of the instrument and a target associated with the medical procedure), and respiration (represented by a deviation of a tip of the scope relative to a direction of movement of the scope).

In some implementations, based on the clinical metrics generated at step 830, the analytics engine may correlate those metrics with an impact to a procedure's success or failure. This may especially be the case for the cloud-based data analytics platform 149 shown in FIG. 1. This is the case because the cloud-based data analytics platform 149 obtains metrics across many different medical procedures. Based on the correlations, an analytics engine (such as analytics engine 141A or analytics engine 141B) may provide pre-operative guidance, intraoperative guidance, or post-operative guidance that can inform the physician. Success can be determined automatically by the medical system. For example, an analytics engine may process the endoscopic video data to detect a needle instrument. In some other implementations, success can be determined manually or semi-automatically. For example, in the manual implementations, a physician may input into the medical system that the percutaneous access procedure was successful or not. As another example, in the semi-automatic implementations, a vision algorithm may detect a needle instrument in the scope camera data and request a user to confirm the success. Success may not necessarily have a binary value but can also have a degree of success based on the location with the papilla that needle instrument has entered, and this can likewise be detected automatically by vision detection, manually detected, or semi-automatically.

While the statistical test for correlation can indicate whether two variables often occur together, it cannot explain whether one causes the other. For example, the analytics engine 141A also may determine that the distance or offset between the scope and a target (such as a calyx or papilla) is statistically correlated with the steadiness of the needle during insertion phase. In this example, a confounding variable (such as the tract length) may have a causal effect on both the target offset and the needle insertion steadiness. Without knowing the causal relationships between various clinical metrics, a medical system can only offer limited inferences or insights about the medical procedure (or the medical system itself) based on the clinical metrics. However, aspects of the present disclosure recognize that causal relationships can be extracted from data using various “causal discovery” techniques (such as the PC, greedy equivalence search (GES), additive noise models (ANM), and NOTEARS causal discovery algorithms, among other examples). In some aspects, the analytics engine 141A (or a component thereof) may further determine causal relationships between the various clinical metrics and generate more refined inferences based on the determined causal relationships.

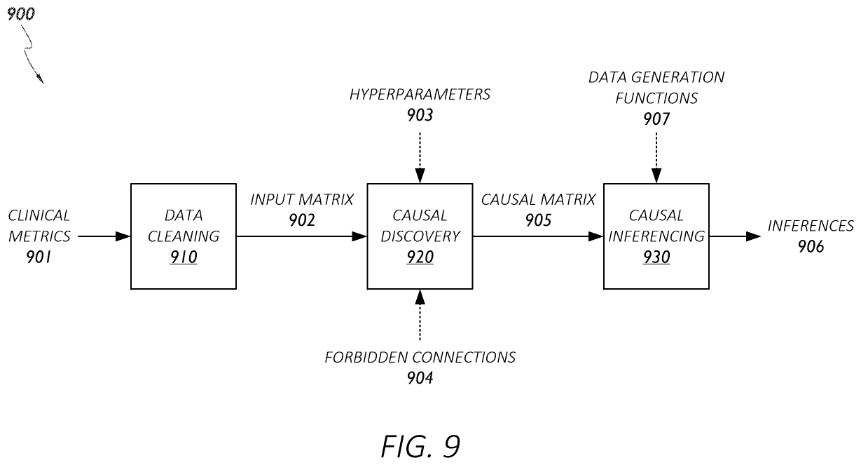

FIG. 9 shows a block diagram of an example inferencing system 900, according to some implementations. In some implementations, the inferencing system 900 may be one example of the analytics engine 141A or the cloud-based data analytics platform 149 of FIG. 1. The inferencing system 900 is configured to receive a set of clinical metrics 901 and generate or produce one or more inferences 906 based on the received metrics 901. The clinical metrics 901 may be associated with any robotically assisted medical procedure. In some implementations, the clinical metrics 901 may be associated with a percutaneous medical procedure (such as any of the procedures described with reference to FIGS. 1-4).

As described with reference to FIG. 8, the clinical metrics 901 may include any phase-specific metrics that can be derived from telemetry data captured or acquired by a medical system during the course of performing the medical procedure. A particular medical procedure (such as PCNL) can be defined or described by a set of clinical metrics that are specific to that procedure. However, the medical system (or the analytics engine) may determine new values for the clinical metrics each time it performs the medical procedure. In other words, each “instance” of the medical procedure may yield new values for the set of clinical metrics 901.

The inferencing system 900 includes a data cleaning component 910, a causal discovery component 920, and a causal inferencing component 930. The data cleaning component 910 is configured to prepare the clinical metrics 901 for causal discovery. In some aspects, the data cleaning component 910 may normalize the values for the clinical metrics 901 to prevent outliers from skewing the dataset. For example, an outlier may be defined as any value (x) that is more than 3 standard deviations (SD) above a mean or average value (μ) for the clinical metric (such as x>μ+3 SD) or less than 3 standard deviations below the mean or average value μ (such as x<μ−3 SD). In some implementations, the data cleaning component 910 may scale or transform such outliers to a maximum value (such as x=μ+3.5 SD if x>μ+3 SD) or a minimum value (such as x=μ−3.5 SD if x<μ−3 SD) associated with a normalized dataset.

In some other aspects, the data cleaning component 910 may impute values for one or more of the clinical metrics 901. For example, in some instances, a medical system may fail to acquire certain telemetry data which may be required for calculating a particular clinical metric. As a result, the set of clinical metrics 901 may be “missing” values from one or more instances of the medical procedure. In some implementations, the data cleaning component 910 may impute values for such clinical metrics based on known values of other clinical metrics 901 in the dataset. For example, the data cleaning component 910 may model each metric with a missing value as a function of other metrics in a round-robin fashion. In some other implementations, the data cleaning component 910 may impute one or more values for a particular clinical metric based on other known values associated with the clinical metric. For example, the data cleaning component 910 may impute a missing value with the mean, median, or mode of the other values associated with the same clinical metric.

Still further, in some aspects, the data cleaning component 910 may scale the values of the clinical metrics 901 to a normalized range of values. As described with reference to FIGS. 1-5, the telemetry data can be captured or acquired by various sensors with different levels or precision or scale. As a result, the values of different clinical metrics 901 may be difficult to compare in their original form. For example, some clinical metrics 901 may have a relatively narrow range of values while some other clinical metrics 901 may have a relatively wide range of values. Moreover, the values associated with some clinical metrics 901 may be significantly higher than the values associated with some other clinical metrics 901 (such as on a scale of tens compared to thousands). In some implementations, the data cleaning component 910 may remove the mean value u from each dataset and scale to unit variance, so that the distributions of the scaled values (z) are centered around the same value(s), where z=z−μ/s. In s some implementations, the data cleaning component 910 may further scale the values of the clinical metrics 901, between 0 and 1, so that each clinical metric has a value greater than or equal to 0 and less than or equal to 1.

The causal discovery component 920 is configured to determine or identify causal relationships between the clinical metrics 901 based on one or more causal discovery techniques. More specifically, the causal discovery component 920 may map the clinical metrics 901 to a connected graph, such as a directed acyclic graph (DAG), based on the determined causal relationships. For example, the causal discovery component 920 may receive the “cleaned” clinical metrics, as an input matrix 902, from the data cleaning component 910 and produce a causal matrix 905 (representing the DAG) that indicates the causal relationships between the metrics. In some implementations, the causal discovery component 920 may perform a causal discovery operation according to a known causal discovery algorithm. Example suitable causal discovery algorithms include the PC algorithm, the greedy equivalence search (GES) algorithm, additive noise models (ANM), or the NOTEARS algorithm.

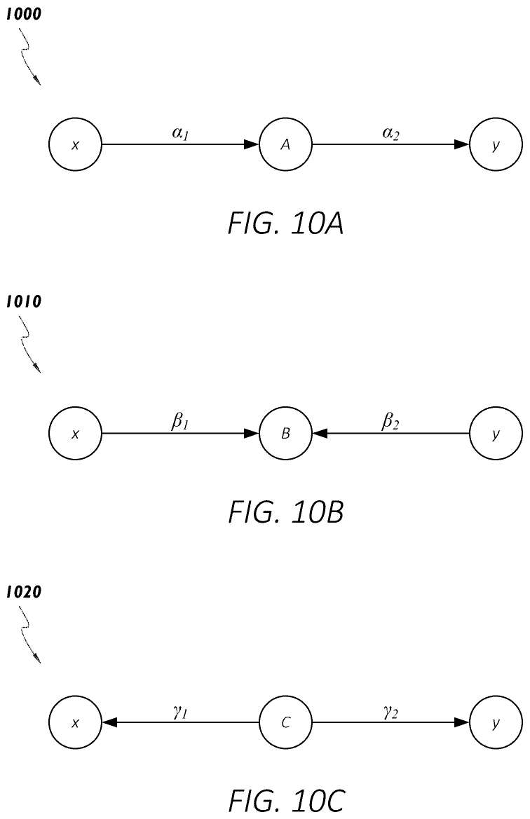

According to the causal Markov condition, causal relationships between random variables result in statistical dependencies among the variables. Thus, if a pair of random variables x and y are statistically correlated, then one of the following must be true: x causes y, y causes x, or there is a confounding variable z that causes both x and y. The converse of this condition is also true. In other words, statistical independencies among random variables imply d-separation between the variables. In a connected graph (such as a DAG), the random variables x and y are directionally separated (or “d-separated”) if a path cannot be traced between x and y (all information flow between x and y is blocked). By contrast, the random variables x and y are directionally connected (or “d-connected”) if a path can be traced between x and y, such as in a “chain,” without traversing a collider. FIG. 10A shows an example chain 1000 between x and y that traverses a random variable set A. In some implementations, a function (α₁) can be determined that relates x to A, and another function (α₂) can be determined that relates A to y. In other words, the function α₁ can be used to recreate A from x, and the function α₂ can be used to recreate y from A.

In a “collider,” the random variables x and y are d-separated by a random variable set B that causes x and y to be independent. FIG. 10B shows an example collider 1010 in which x and y are d-separated by the random variable set B. As shown in FIG. 10B, x and y are both causal parents of B. Thus, by conditioning either x or y on the set of variables B (thus removing the impact or effect of B), a path can be traced between x and y. As a result, the random variables x and y become d-connected (or “conditionally dependent”). In some implementations, a function (β₁) can be determined that relates x to B, and another function (β₂) can be determined that relates y to B. In other words, the function β₁ can be used to recreate B from x, and the function β₂ can be used to recreate B from y.

In a “fork,” the random variables x and y are d-connected by a random variable set C that causes x and y to be dependent. FIG. 10C shows an example fork 1020 in which x and y are d-connected by the random variable set C. As shown in FIG. 10C, x and y are both causal children of C. Thus, by conditioning either x or y on the set of variables C (thus removing the impact or effect of C), a path cannot be traced between x and y. As a result, the random variables x and y become d-separated (or “conditionally independent”). In some implementations, a function (γ₁) can be determined that relates C to x, and another function (γ₂) can be determined that relates C to y. In other words, the function γ₁ can be used to recreate x from C, and the function γ₂ can be used to recreate y from C.

By applying the principles above, the causal discovery component 920 may use statistical tests for conditional independence to determine whether pairs of clinical metrics 901 are causally related. For example, the causal discovery component 920 may begin the causal discovery operation with the assumption that the clinical metrics 901 are all related to one another (such that each of the clinical metrics is connected to each of the remaining clinical metrics in a connected graph). If the causal discovery component 920 determines that a pair of clinical metrics are statistically independent (or uncorrelated), the causal discovery component 920 may subsequently conclude that the pair of metrics are not causally related (or that the pair of metrics are d-separated) and remove the edge connecting the metrics in the connected graph. The causal discovery component 920 may iteratively test for statistical independence on two clinical metrics by conditioning on a different set of variables with each iteration until the connections no longer change.

In some implementations, the causal discovery component 920 may further receive one or more forbidden connections 904 indicating clinical metrics that cannot be related for various reasons. More specifically, the forbidden connections 904 may be used to reduce or limit the number of connections in the connected graph. The forbidden connections 904 may be determined or otherwise specified based on other information or metadata associated with the clinical metrics 901. In some implementations, one or more of the forbidden connections 904 may be associated with temporal information indicating time dependencies among the clinical metrics 901 that are already known (such as described with reference to FIGS. 5-8). In such implementations, the temporal information may indicate one or more forbidden connections 904 between clinical metrics 901 that cannot be related based on their temporal relationships (or lack thereof).

The causal discovery component 920 may repeat the test for statistical independence on a different pair of clinical metrics until the causal discovery operation converges on a “skeleton” of the DAG. For example, the skeleton may include all of the connections between the nodes but not the directionality of the edges. The causal discovery component 920 may determine the directionality of the edges in the DAG (represented by the causal matrix 905) based, at least in part, on the rules described above. For example, if an edge connecting variables x and y was removed when the test for statistical independence was conditioned on a variable j, the arrows connecting the variable j to each of the variables x and y must point towards the variable j (according to the rule for colliders). In some implementations, the causal discovery component 920 may further leverage temporal information to determine the directionality of the edges in the DAG. For example, if variable x is known to occur (temporally) before variable y, then variable y cannot be the cause of variable x. Thus, if the variables x and y are connected by an edge, the arrow must point from x to y.

Aspects of the present disclosure recognize that some causal discovery algorithms can be tuned using one or more hyperparameters 903. For example, the PC algorithm has a tunable hyperparameter that represents a probability of rejecting a null hypothesis when the null hypothesis is true. More specifically, lower values of a are associated with more rigorous causal discovery, which results in fewer but stronger connections between the nodes of the DAG. By contrast, higher values of a are associated with less rigorous causal discovery, which results in greater but weaker connections between the nodes of the DAG. Thus, the hyperparameters 903 may be selected based on various factors, such as sample size, noise in the data, and requirements for confidence in each node. In some other implementations, the causal discovery component 920 may perform the casual discovery operation according to a new casual discovery algorithm that is optimized or otherwise preconfigured for determining causal relationships between clinical metrics associated with a robotically assisted medical procedure.

In some implementations, the causal matrix 905 (or DAG) generated by the causal discovery component 920 may be validated or refined by one or more subject matter experts (SMEs) before being used for causal inferencing. The SMEs may fine-tune the causal matrix 905 based on their knowledge of the medical system or the medical procedure. For example, an SME may remove any causal connections between variables which the SME knows cannot be causally related. Aspects of the present disclosure recognize that some causal matrices may be relatively small or simple (depending on the medical procedure or a particular portion of the medical procedure to be represented in the DAG), which may not require substantial processing resources to generate. Thus, in some other implementations, an SME (rather than the causal discovery component 920) may create a causal matrix based on the clinical metrics 901. In such implementations, the SME-created causal matrix may be provided directly to the causal inferencing component 930 in addition to, or in lieu of, the causal matrix 905.

The DAG represented by the causal matrix 905 indicates the causal structure and statistical relationships between the clinical metrics 901. As such, the DAG can be used to perform “do-calculus” or draw inferences about the effects of a hypothetical intervention (such as an intervention that did not occur during an actual procedure) using only observational data. Graphical causal models (GCMs) can provide additional insights about the dataset based on the DAG and knowledge of the data generation process. More specifically, a GCM can be derived based on a causal mechanism that defines the data distribution of each node in the DAG given the data distribution of its parent node(s). The causal mechanism can be any function 907 (also referred to as a “data generation function”) that relates any given node Xᵢ in the DAG to its causal parents PAᵢ (such as any of the functions α₁, α₂, β₁, β₂, γ₁, and/or γ₂ of FIGS. 10A-10C).

Example suitable GCMs include probabilistic causal models (PCMs), structural causal models (SCMs), and invertible structural causal models (ISCMs). In a PCM, root nodes (which have no causal parents) are modeled by their own probability distributions, and non-root nodes Xᵢ are modeled by a conditional probability distribution based on their causal parents PAᵢ. In an SCM, any given node Xᵢ can be modeled as a function of PAᵢ and noise Nᵢ, which represents the unmeasured or unidentified parents of Xᵢ (not included in the DAG). An ISCM is similar to an SCM, except that the noise Nᵢ can be constructed from the observed data. In other words, the noise component Nᵢ can be derived by reversing the function Fᵢ associated with the SCM.

The causal inferencing component 930 is configured to generate one or more inferences 906 based on the causal matrix 905 (or DAG) and/or a GCM associated therewith. The causal inferencing component 930 may generate the inferences 906 using any known causal inferencing techniques. Example suitable causal inferencing techniques may include causal effect estimation and counterfactual calculations, among other examples. For causal effect estimation, the causal inferencing component 930 may identify a target estimand based on the causal matrix 905, estimate a causal effect on the target estimand, and refute the estimate (or verify the robustness of the estimate to assumption violations). For counterfactual calculations, the causal inferencing component 930 also may estimate the outcome of a given variable under different conditions for other variables (such as in response to “what if” queries). For example, given a dataset indicating that a particular outcome (Z) was observed for a first variable (z) when a second variable (x) was set to a particular value (X), the causal inferencing component 930 can leverage an ISCM associated with the causal matrix 905 to estimate what would have happened to the outcome Z if the second variable x had a different value (Y).

The inferences 906 may be output to, or displayed on, an electronic device (such as the display 142 or a third-party computer 147 of FIG. 1) so that the inferences 906 can be viewed or otherwise utilized by a user or operator of the medical system (such as a physician or technician) or any person or entity associated with the medical system or the medical procedure. In some implementations, the inferences 906 may include a visual representation of the DAG for display on the electronic device. For example, the DAG may indicate which clinical variable has a casual effect on the greatest number of other clinical variables or the greatest causal effect on the success of the medical procedure. Thus, research and development (R&D) teams can use the DAG to identify specific areas of the medical system that can be improved while yielding the maximum return on investment.

In some other implementations, the inferences 906 may highlight or otherwise indicate one or more clinical metrics that caused a given instance of the medical procedure to fail. For example, the causal inferencing component 930 may determine, based on the DAG, which clinical metrics have a causal effect (as well as the causal effect such metrics have) on the success of the medical procedure or on other clinical metrics associated with the DAG. The inferencing component 930 may further determine, for any set of clinical metrics having a causal relationship to the outcome of the procedure (also referred to as “causal metrics”), a range of values that result in a successful medical procedure. The causal inferencing component 930 may compare each range of values to new values for the causal metrics (associated with the given instance of the medical procedure) to identify areas for improvement. Physicians may use such inferences 906 to focus their time and efforts on improving a narrow set of skills or techniques, or achieving a target range for certain clinical metrics, that may have the greatest impact on their performance of the medical procedure.

In some other implementations, the inferences 906 may highlight or otherwise indicate one or more clinical metrics that have a causal effect on a problem associated with a complaint investigation. For example, if the complaint indicates a problem with a given clinical metric, the causal inferencing component 930 may determine, based on the DAG, which of the other clinical metrics have a causal effect on the given metric. Such inferences 906 may be used to expedite or automate complaint investigations. For example, if a physician alleges that they were unable to access a kidney stone using the medical system, the causal inferencing component 930 may identify a narrow set of clinical metrics that could have caused the problem. A technician could then review the case values for those causal metrics to diagnose the issue.

Still further, in some implementations, the one or more inferences 906 may include a simulated randomized controlled trial (RCT) using only observational data. For example, the causal inferencing component 930 may use the DAG, identification, estimation, and refutation to predict the outcome of a medical procedure under varying conditions (such as by setting a given “treatment” variable to a predetermined value and predicting the outcome under such conditions). By simulating RCTs, aspects of the present disclosure may allow R&D teams to test new features for the medical system on real world data and make product claims based on purely observational data and without the expense of clinical trials.

As described above, the success or outcome of a medical procedure depends on various factors, including patient anatomy, physician skill, system performance, and workflow decisions. By generating clinical metrics based on robotic telemetry data, aspects of the present disclosure can measure and quantify such features in a manner suitable for statistical analysis. By performing causal discovery on the clinical metrics associated with a medical procedure, aspects of the present disclosure can further identify causal relationships between the clinical metrics which may not be evident otherwise (such as based solely on statistical correlations).

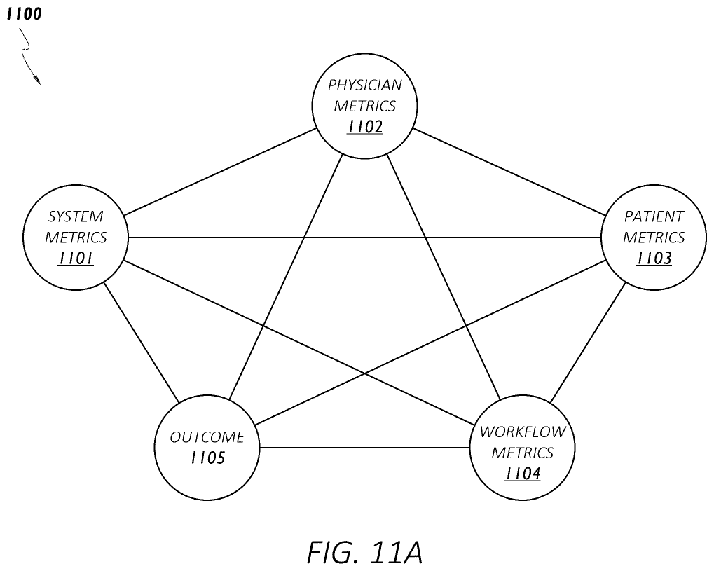

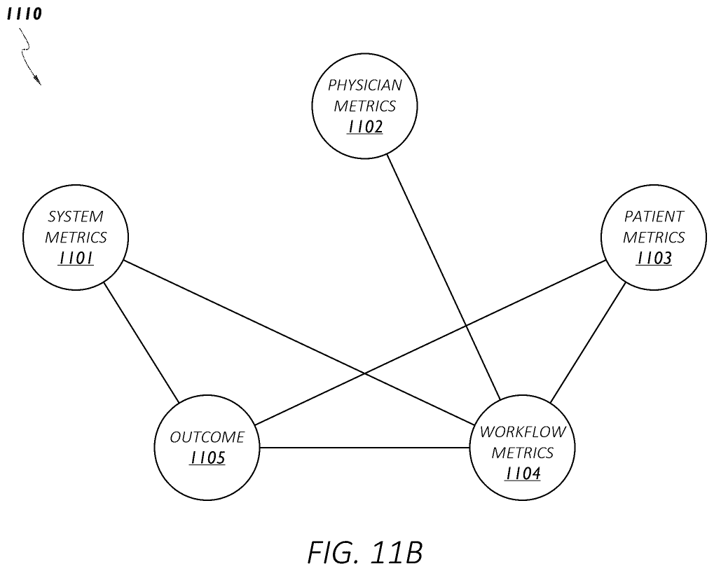

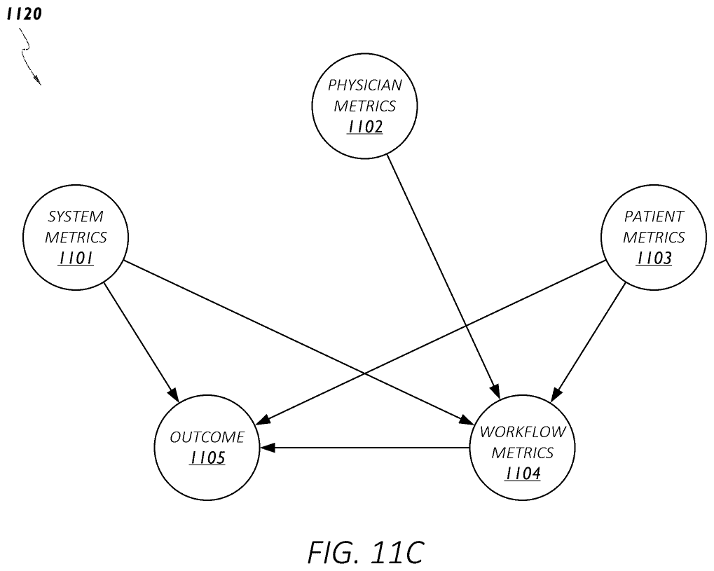

FIG. 11A shows an example fully connected graph 1100 of clinical metrics that can be provided as input to a causal discovery system. In some implementations, the causal discovery system may be one example of the causal discovery component 920 of FIG. 9. With reference to FIG. 9, the connected graph 1100 may be one example of the input matrix 902. For example, the rows and columns of the input matrix 902 may indicate the connections between the various clinical metrics.

In the example of FIG. 11A, the connected graph 1100 depicts a mapping of system metrics 1101, physician metrics 1102, patient metrics 1103, workflow metrics 1104, and am outcome 1105. Although depicted as a single node in the connected graph 1100, each set of system metrics 1101, physician metrics 1102, patient metrics 1103, or workflow metrics 1104 may include one or more clinical metrics associated therewith. For example, the system metrics 1101 may include one or more metrics based on force values associated with tools or instruments used in the medical procedure, the physician metrics 1102 may include one or more metrics based on sensor values indicating the steadiness or control of the physician in navigating the medical tools or instruments, the patient metrics 1103 may include one or more metrics based on sensor values indicating characteristics of the patient's anatomy (such as respiration, tract length, or anatomical angles), and the workflow metrics 1104 may include one or more metrics indicating any sequence of actions taken (or not taken) by the physician in performing the medical procedure. The outcome 1105 may include a procedural outcome (such as whether percutaneous access was successful) or a clinical outcome (such as whether a kidney stone was successfully removed).

As shown in FIG. 11A, each set of metrics 1101-1105 is initially presumed to be statistically correlated with each of the remaining sets of metrics (as shown by the connections in the graph 1100). Further, clinical metrics within a given set (not shown for simplicity) are also presumed to be statistically correlated with one another. During a causal discovery operation, the causal discovery system may iteratively test the statistical dependencies of each pair of clinical metrics conditioned on different sets of variables until the connected graph 1100 converges on a skeleton of the DAG (such as described with reference to FIG. 9). For example, if the causal discovery system determines that a pair of clinical metrics are statistically independent (or uncorrelated), the causal discovery system may subsequently conclude that the pair of metrics are not causally related and remove the edge connecting the metrics in the connected graph 1100. In some implementations, the causal discovery system may leverage temporal information (or other information) associated with the clinical metrics to reduce or limit the number of connections in the connected graph 1100 (such as via forbidden connections).

FIG. 11B shows an example causal graph 1110 indicating various casual dependencies between the clinical metrics shown in FIG. 11A. More specifically, the example causal graph 1110 represents an intermediate output of the causal discovery operation performed on the connected graph 1100.

In the example of FIG. 11B, the causal discovery system has finished “pruning” the edges of the connected graph 1100 to produce the causal graph 1110. Accordingly, the causal graph 1110 includes only a subset of the edges of the fully connected graph 1100. However, each of the edges of the causal graph 1110 depicts a causal relationship between two nodes (or sets of metrics). According to the causal graph 1110 of FIG. 11B, the system metrics 1101 are causally related to the workflow metrics 1104 and the outcome 1105; the physician metrics 1102 are causally related to the workflow metrics 1104; the patient metrics 1103 are causally related to the workflow metrics 1104 and the outcome 1105; and the workflow metrics 1104 are also causally related to the outcome 1105.

In some implementations, the causal graph 1110 may represent a skeleton of the DAG that the causal discovery system is attempting to discover. Thus, while the edges of the causal graph 1110 represent the edges of the DAG, the directionality of each edge is still unknown. The causal discovery system may determine the directionality of the edges in the causal graph 1110 based, at least in part, on the rules associated with the causal Markov condition (such as described with reference to FIG. 9). In some implementations, the causal discovery system may further leverage temporal information (or other known information) associated with the clinical metrics to determine the directionality of the edges in the causal graph 1110. For example, because aspects of the workflow cannot affect inherent characteristics of the system, the physician, or the patient, none of the workflow metrics 1104 can be the cause of any of the system metrics 1101, physician metrics 1102, or patient metrics 1103. In other words, the direction of the edges connecting each of the metrics 1101-1103 to the workflow metric 1104 must point in the direction of the workflow metric 1104.

FIG. 11C shows an example DAG 1120 indicating various causal effects of the clinical metrics shown in FIG. 11A. More specifically, the example DAG 1120 represents a final output of the causal discovery operation performed on the connected graph 1100.

In the example of FIG. 11C, the causal discovery system has finished determining the directionality of the edges of the causal graph 1110 to produce the DAG 1120. Accordingly, the edges of the DAG 1120 are the same as the edges of the causal graph 1110. However, each of the edges of the DAG 1120 depicts a cause-and-effect relationship between two nodes (or sets of clinical metrics), as indicated by the direction of the arrows. According to the DAG 1120 of FIG. 11C, the system metrics 1101 have a causal effect on the workflow metrics 1104 and the outcome 1105; the physician metrics 1102 have a causal effect on the workflow metrics 1104; the patient metrics 1103 have a causal effect on the workflow metrics 1104 and the outcome 1105; and the workflow metrics 1104 have a causal effect on the outcome 1105.

As described with reference to FIGS. 11A-11C, the causal discovery operation can untangle the causal relationships between any clinical metrics associated with a given dataset. Although the DAG 1120 shows the general, high-level causal relationships between various types of clinical metrics, the interconnections between individual metrics may be much more complex at lower levels. For example, in actual implementations, clinical metrics may not be categorized as system metrics 1101, physician metrics 1102, patient metrics 1103, or workflow metrics 1104. Rather, the causal discovery operation may treat each clinical metric as an individual random variable to be conditioned on various other sets of random variables. Accordingly, the number of connections in a given DAG may be significantly greater than what is depicted in FIG. 11C.

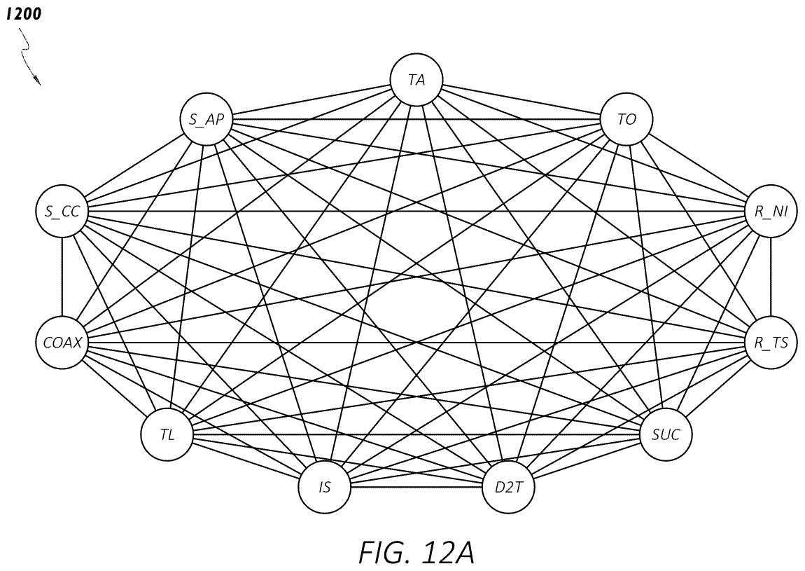

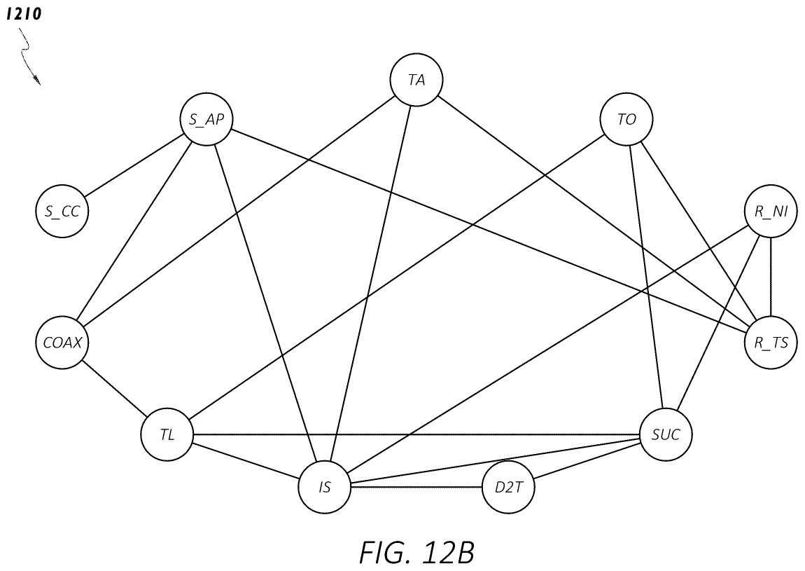

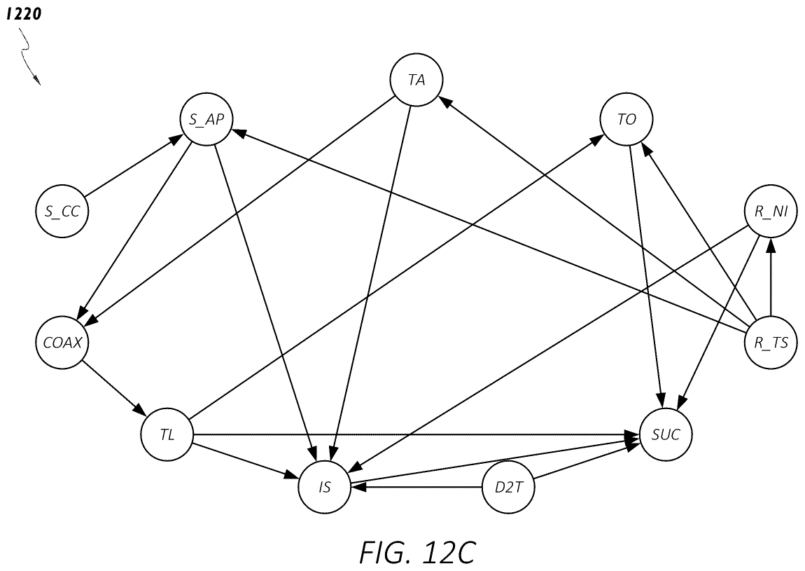

FIG. 12A shows another example fully connected graph 1200 of clinical metrics that can be provided as input to a causal discovery system, according to some implementations. In some implementations, the causal discovery system may be one example of the causal discovery component 920 of FIG. 9. With reference to FIG. 9, the connected graph 1200 may be one example of the input matrix 902. For example, the rows and columns of the input matrix 902 may indicate the connections between the various clinical metrics.

In the example of FIG. 12A, the clinical metrics are associated with a urological procedure. More specifically, the connected graph 1200 depicts a mapping of 11 clinical metrics: respiration during a target selection phase (R_TS), respiration during a needle insertion phase (R_NI), target offset (TO), target angle (TA), anterior/posterior scope angle (S_AP), cranial/caudal scope angle (S_CC), coaxiality (COAX), tract length (TL), insertion steadiness (IS), distance to target (D2T), and success of the medical procedure (SUC). However, the example metrics are shown for purposes of illustration only. In some other implementations, the connected graph 1200 may include fewer or more clinical metrics, or different clinical metrics, than those depicted in FIG. 12A.

As described with reference to FIG. 8, the R_TS and R_NI metrics represent a deviation of a scope tip relative to a direction of movement of the scope (during respective phases of the medical procedure); the TO metric indicates a distance between a tip of the scope and a EM-target that the user created intra-operatively; the TA metric indicates a difference in angle between the position of the scope when tagging a target location and the position of the scope at park position; the S_AP metric indicates an anterior-posterior angle of the scope; the S_CC metric indicates a cranial-caudal angle of the scope; the COAX metric indicates the angle between the needle instrument and scope; the TL metric indicates a distance between a tip of the instrument and a desired location (during a site selection phase); the IS metric represents a deviation of a tip of the instrument relative to a principal insertion direction; the D2T metric indicates the shortest distance between a tip of the instrument and a target associated with the medical procedure (during the needle insertion phase); and the SUC metric is a binary indicator of whether the procedure was successful (such as whether the physician gained access to the patient's kidney).

As shown in FIG. 12A, each of the clinical metrics R_TS, R_NI, TO, TA, S_AP, S_CC, COAX, TL, IS, D2T, and SUC is initially presumed to be statistically correlated with each of the other clinical metrics in the set (as shown by the connections in the graph 1200). In other words, each of the clinical metrics R_TS, R_NI, TO, TA, S_AP, S_CC, COAX, TL, IS, D2T, and SUC is shown connected to 10 other clinical metrics in the connected graph 1200. During a causal discovery operation, the causal discovery system may iteratively test the statistical dependencies of each pair of clinical metrics conditioned on different sets of variables until the connected graph 1200 converges on a skeleton of the DAG (such as described with reference to FIG. 9). For example, if the causal discovery system determines that a pair of clinical metrics are statistically independent (or uncorrelated), the causal discovery system may subsequently conclude that the pair of metrics are not causally related and remove the edge connecting the metrics in the connected graph 1200. In some implementations, the causal discovery system may leverage temporal information (or other information) associated with the clinical metrics to reduce or limit the number of connections in the connected graph 1200 (such as via forbidden connections).

FIG. 12B shows another example causal graph 1210 indicating various casual dependencies between the clinical metrics shown in FIG. 12A. More specifically, the example causal graph 1210 represents an intermediate output of the causal discovery operation performed on the connected graph 1200.

In the example of FIG. 12B, the causal discovery system has finished pruning the edges of the connected graph 1200 to produce the causal graph 1210. Accordingly, the causal graph 1210 includes only a subset of the edges of the fully connected graph 1200. However, each of the edges of the causal graph 1210 depicts a causal relationship between two nodes (or metrics). According to the causal graph 1210 of FIG. 12B, the R_TS metric is causally related to each of the R_NI, TO, TA, and S_AP metrics; the R_NI metric is also causally related to each of the IS and SUC metrics; the TO metric is also causally related to each of the TL and SUC metrics; the TA metric is also causally related to each of the COAX and IS metrics; the S_AP metric is also causally related to each of the S_CC, COAX, and IS metrics; the COAX metric is also causally related to the TL metric; the TL is also causally related to each of the IS and SUC metrics; the IS metric is also causally related to each of the D2T and SUC metrics; and the D2T metric is also causally related to the SUC metric.

In some implementations, the causal graph 1210 may represent a skeleton of the DAG that the causal discovery system is attempting to discover. Thus, while the edges of the causal graph 1210 represent the edges of the DAG, the directionality of each edge is still unknown. The causal discovery system may determine the directionality of the edges in the causal graph 1210 based, at least in part, on the rules associated with the causal Markov condition (such as described with reference to FIG. 9). For example, if an edge connecting the S_CC and R_TS metrics was removed when the test for statistical independence was conditioned on the S_AP metric, the direction of the edges connecting the S_AP metric to each of the S_CC and R_TS metrics must point towards the S_AP metric (according to the rule for colliders). In some implementations, the causal discovery system may further leverage temporal information (or other information) associated with the clinical metrics to determine the directionality of the edges in the causal graph 1210. For example, because the R_TS metric is known to occur before the R_NI metric (by virtue of the fact that the target select phase precedes the needle insertion phase), then the R_NI metric cannot be the cause of the R_TS metric. In other words, the direction of the edge connecting the R_NI metric to the R_TS metric must point towards the R_NI metric.

FIG. 12C shows another example DAG 1220 indicating various causal effects of the clinical metrics shown in FIG. 12A. More specifically, the example DAG 1220 represents a final output of the causal discovery operation performed on the connected graph 1200.

In the example of FIG. 12C, the causal discovery system has finished determining the directionality of the edges of the causal graph 1210 to produce the DAG 1220. Accordingly, the edges of the DAG 1220 are the same as the edges of the causal graph 1210. However, each of the edges of the DAG 1220 depicts a cause-and-effect relationship between two nodes (or metrics), as indicated by the direction of the arrows. According to the DAG 1220 of FIG. 12C, the R_TS metric has a causal effect on each of the R_NI, TO, TS, and S_AP metrics; the R_NI metric has a causal effect on each of the IS and SUC metrics; the TO metric has a causal effect on the SUC metric; the TA metric has a causal effect on each of the COAX and IS metrics; the S_AP metric also has a causal effect on each of the COAX and IS metrics; the S_CC metric has a causal effect on the S_AP metric; the COAX metric has a causal effect on the TL metric; the TL metric has a causal effect on each of the TO, IS, and SUC metrics; the IS metric has a causal effect on the SUC metric; and the D2T metric has a causal effect on each of the IS and SUC metrics.

As described with reference to FIG. 9, the DAG 1220 provides much greater insight about the medical system (and the medical procedure) than statistical correlations alone. For example, the DAG 1220 can be used to optimize the allocation of R&D resources, automate complaint investigations, provide personalized feedback and case review that is narrowly tailored or curated for a particular physician, and simulate clinical trials or RCTs (as described with reference to FIG. 9).

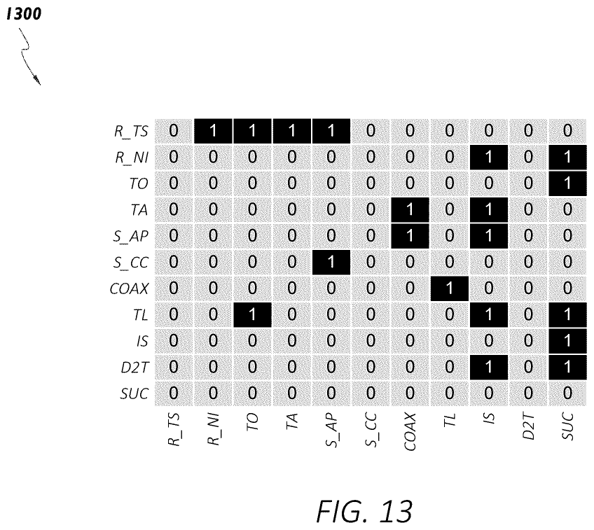

FIG. 13 shows an example matrix 1300 representing the DAG 1220 depicted in FIG. 12C. In some implementations, the matrix 1300 may be one example of the causal matrix 905 of FIG. 9. More specifically, the example matrix 1300 is another visual representation of the DAG 1220.

As described with reference to FIG. 9, a DAG can be represented by a matrix, having rows and columns associated with different clinical metrics, where the intersection of each row and column indicates a presence (“1”) or absence (“0”) of an edge in the DAG 1220. As shown in FIG. 13, each row of the matrix 1300 is labeled with a respective one of the clinical metrics R_TS, R_NI, TO, TA, S_AP, S_CC, COAX, TL, IS, D2T, and SUC and each column of the matrix 1300 is also labeled with a respective one of the clinical metrics R_TS, R_NI, TO, TA, S_AP, S_CC, COAX, TL, IS, D2T, and SUC. In the example of FIG. 13, the matrix 1300 can be read or interpreted from row to column to identify cause and effect, where the clinical metric associated with a given row has a causal effect on the clinical metric associated with a given column. For example, the 1's in the upper-most row of the matrix 1300 (associated with the R_TS metric) indicate that the R_TS metric has a causal effect on the R_NI, TO, TA, and S_AP metrics. On the other hand, the 0's in the upper-most row of the matrix 1300 indicates that the R_TS metric does not have a causal effect on any of the S_CC, COAX, TL, IS, D2T, or SUC metrics.

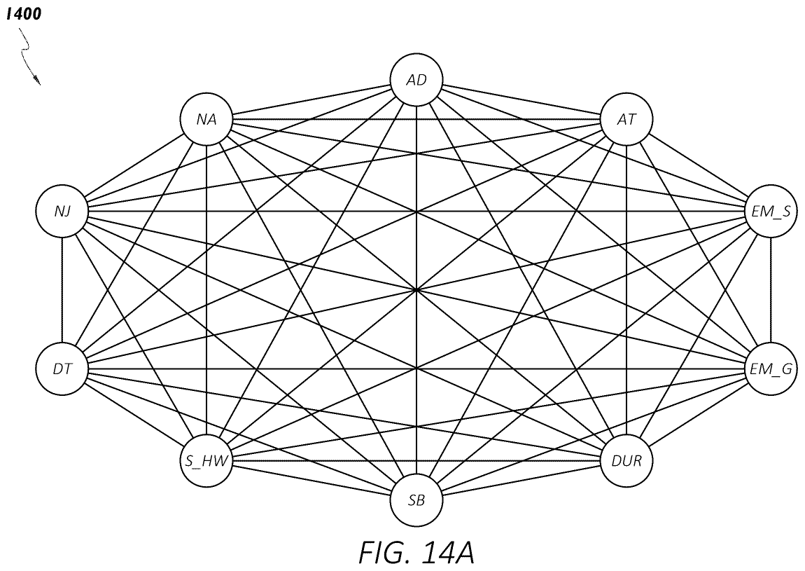

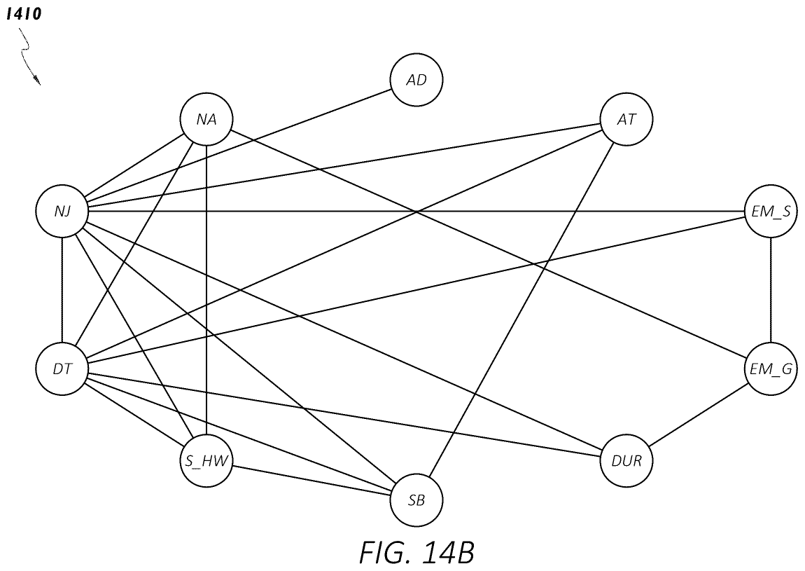

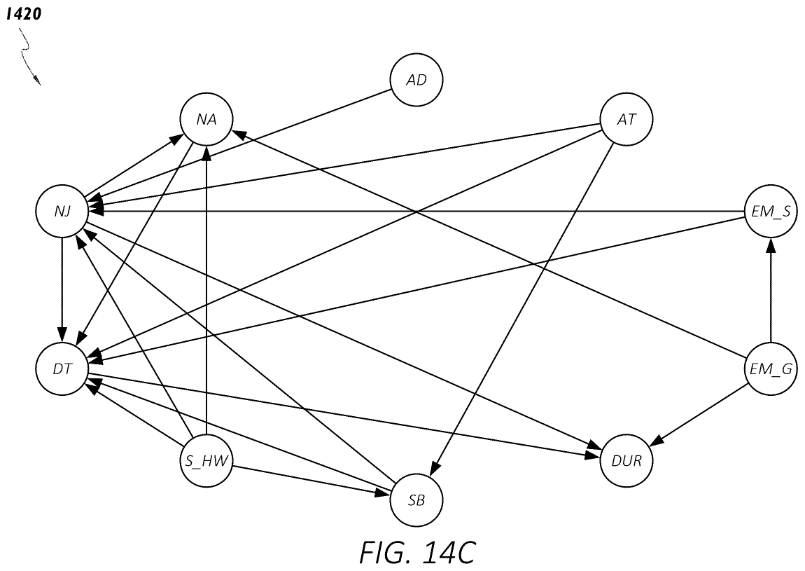

FIG. 14A shows another example fully connected graph 1400 of clinical metrics that can be provided as input to a causal discovery system. In some implementations, the causal discovery system may be one example of the causal discovery component 920 of FIG. 9. With reference to FIG. 9, the connected graph 1400 may be one example of the input matrix 902. For example, the rows and columns of the input matrix 902 may indicate the connections between the various clinical metrics.

In the example of FIG. 14A, the clinical metrics are associated with a bronchoscopy procedure. More specifically, the connected graph 1400 depicts a mapping of 10 clinical metrics: EM generator model (EM_G), scope EM quality (EM_S), anatomical tortuosity (AT), average airway diameter (AD), navigation accuracy (NA), navigation jumps (NJ), active driving time (DT), scope hardware limitations (S_HW), scope buckling faults (SB), and duration (DUR). However, the example metrics are shown for purposes of illustration only. In some other implementations, the connected graph 1400 may include fewer or more clinical metrics, different clinical metrics, and/or different connections between the clinical metrics than those depicted in FIG. 14A.

The EM_G metric characterizes the hardware used for generating an EM field (which defines the EM sensor space for localizing tools and/or objects inside an anatomy); the EM_S metric indicates the quality of the EM data acquired from one or more EM sensors disposed on a scope (such as in response to the EM field); the AT metric indicates the shape of a luminal network associated with the anatomy (such as how tortuous of a path the scope must navigate through the bronchial network); the AD metric indicates the average diameter of the anatomical lumens (or airways); the NA metric indicates the accuracy of navigational guidance provided by the medical system to a user driving or manipulating the scope; the NJ metric indicates a number and/or measure of jumps or sudden movements in the navigational guidance; the DT metric indicates how long the user was actively driving or controlling the scope; the S_HW metric characterizes the physical limitations of the scope hardware (such as how often a user articulates the scope to its maximum limit); the SB metric indicates a number of faults detected due to buckling of the scope; and the DUR metric indicates an overall duration of the procedure (which may be indicative of a successful or unsuccessful procedure).

In the example of FIG. 14A, each of the clinical metrics AD, AT, EM_S, EM_G, DUR, SB, S_HW, DT, NJ, and NA is initially presumed to be statistically correlated with each of the other clinical metrics in the set (as shown by the connections in the graph 1400). In other words, each of the clinical metrics AD, AT, EM_S, EM_G, DUR, SB, S_HW, DT, NJ, and NA is shown connected to 9 other clinical metrics in the connected graph 1400. However, in some other implementations (such as based on other causal discovery techniques), the connections between the clinical metrics may differ from what is shown in FIG. 14A. During a causal discovery operation, the causal discovery system may iteratively test the statistical dependencies of each pair of clinical metrics conditioned on different sets of variables until the connected graph 1400 converges on a skeleton of the DAG (such as described with reference to FIG. 9). In some implementations, the causal discovery system may leverage temporal information (or other information) associated with the clinical metrics to reduce or limit the number of connections in the connected graph 1400 (such as via forbidden connections).

FIG. 14B shows another example causal graph 1410 indicating various casual dependencies between the clinical metrics shown in FIG. 14A. More specifically, the example causal graph 1410 represents an intermediate output of the causal discovery operation performed on the connected graph 1400.

In the example of FIG. 14B, the causal discovery system has finished pruning the edges of the connected graph 1400 to produce the causal graph 1410. Accordingly, the causal graph 1410 includes only a subset of the edges of the fully connected graph 1400. However, each of the edges of the causal graph 1410 depicts a causal relationship between two nodes (or metrics). According to the causal graph 1410 of FIG. 14B, the AD metric is causally related to NJ metric; the AT metric is causally related to each of the NJ, DT, and SB metrics; the EM_S metric is causally related to each of the NJ, DT, and EM_G metrics; the EM_G metric is also causally related to each of the NA and DUR metrics; the DUR metric is also causally related to each of the NJ and DT metrics; the SB metric is also causally related to each of the NJ, DT, and S_HW metrics; the S_HW metric is also causally related to each of the DT, NJ, and NA metrics; the DT metric is also causally related to each of the NJ and NA metrics; and the NJ metric is also causally related to the NA metric.

In some implementations, the causal graph 1410 may represent a skeleton of the DAG that the causal discovery system is attempting to discover. As described with reference to FIG. 12B, the causal discovery system may determine the directionality of the edges in the causal graph 1410 based, at least in part, on the rules associated with the causal Markov condition (such as described with reference to FIG. 9). In some implementations, the causal discovery system may further leverage temporal information (or other information) associated with the clinical metrics to determine the directionality of the edges in the causal graph 1410.

FIG. 14C shows another example DAG 1420 indicating various causal effects of the clinical metrics shown in FIG. 14A. More specifically, the example DAG 1420 represents a final output of the causal discovery operation performed on the connected graph 1400.

In the example of FIG. 14C, the causal discovery system has finished determining the directionality of the edges of the causal graph 1410 to produce the DAG 1420. Accordingly, each of the edges of the DAG 1420 depicts a cause-and-effect relationship between two nodes (or metrics), as indicated by the direction of the arrows. According to the DAG 1420 of FIG. 14C, the AD metric has a causal effect on the NJ metric; the AT metric has a causal effect on each of the NJ, DT, and SB metrics; the EM_S metric has a causal effect on each of the NJ and DT metrics; the EM_G metric has a causal effect on each of the EM_S, NA, and DUR metrics; the SB metric also has a causal effect on each of the NJ and DT metrics; the S_HW metric has a causal effect on each of the SB, NA, NJ, and DT metrics; the DT metric has a causal effect on the DUR metric; the NJ metric has a causal effect on each of the DT, DUR, and NA metrics; and the NA metric has a causal effect on the DT metric.

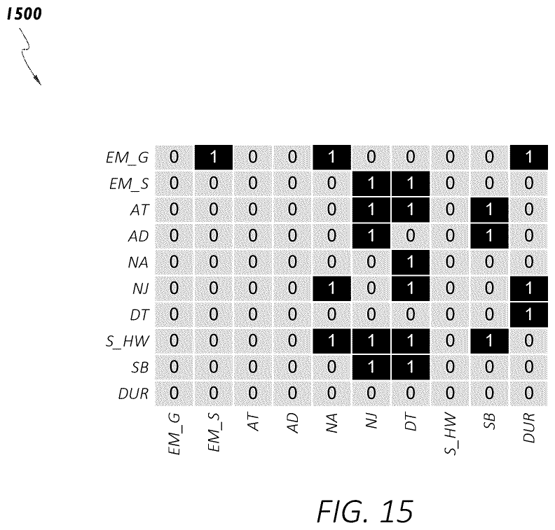

FIG. 15 shows an example matrix 1500 representing the DAG 1420 depicted in FIG. 14C. In some implementations, the matrix 1500 may be one example of the causal matrix 905 of FIG. 9. More specifically, the example matrix 1500 is another visual representation of the DAG 1420.

As shown in FIG. 15, each row of the matrix 1500 is labeled with a respective one of the clinical metrics EM_G, EM_S, AT, AD, NA, NJ, DT, S_HW, SB, and DUR and each column of the matrix 1500 is also labeled with a respective one of the clinical metrics EM_G, EM_S, AT, AD, NA, NJ, DT, S_HW, SB, and DUR. The intersection of each row and column indicates a presence (“1”) or absence (“0”) of an edge in the DAG 1420. As described with reference to FIG. 13, the matrix 1500 can be read or interpreted from row to column to identify cause and effect, where each “1” in a given row indicates that the clinical metric associated with the row has a causal effect on the clinical metric associated with the column in which the “1” resides.

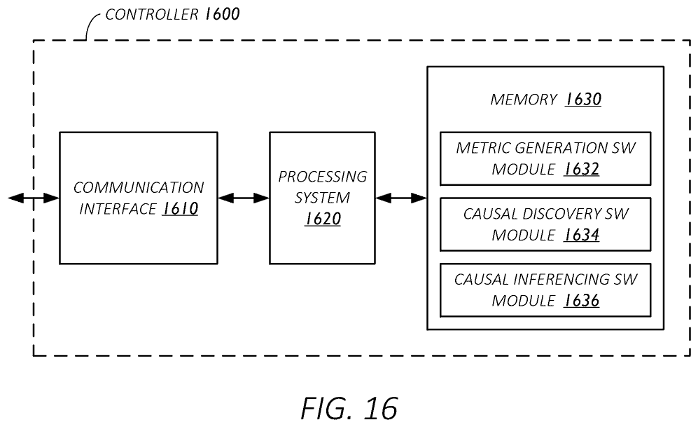

FIG. 16 shows a block diagram of an example causal inferencing system 1600, according to some implementations. In some implementations, the causal inferencing system 1600 may be one example of the inferencing system 900 of FIG. 9 or any of the analytics engine 141A or the cloud-based data analytics platform 149 of FIG. 1. More specifically, the causal inferencing system 1600 is configured to generate one or more inferences associated with a medical system based on data or telemetry about a procedure performed by the medical system.

The causal inferencing system 1600 includes a communication interface 1610, a processing system 1620, and a memory 1630. The communication interface 1610 is configured to communicate with one or more components of the medical system. In some implementations, the communication interface 1610 may receive data associated with the procedure performed by the medical system. In some aspects, the data may include telemetry generated by a robotic system. In some implementations, the telemetry may indicate one or more poses, movements, or actuations of a robotic apparatus (such as a robotic arm) associated with the robotic system. In some other implementations, the telemetry may indicate one or more poses of an instrument or scope associated with the robotic system.

The memory 1630 may include a non-transitory computer-readable medium (including one or more nonvolatile memory elements, such as EPROM, EEPROM, Flash memory, or a hard drive, among other examples) that may store the following software (SW) modules: a metric generation SW module 1632 to determine a plurality of clinical metrics associated with the procedure based at least in part on the received data; a causal discovery SW module 1634 to map the plurality of clinical metrics to a directed acyclic graph (DAG) based on a causal discovery operation that identifies one or more causal relationships between the plurality of clinical metrics; and a causal inferencing SW module 1636 to generate one or more inferences associated with the medical system based at least in part on the DAG.

The processing system 1620 may include any suitable one or more processors capable of executing scripts or instructions of one or more software programs stored in the causal inferencing system 1600 (such as in the memory 1630). For example, the processing system 1620 may execute the metric generation SW module 1632 to determine a plurality of clinical metrics associated with the procedure based at least in part on the received data. The processing system 1620 also may execute the causal discovery SW module 1634 to map the plurality of clinical metrics to a DAG based on a causal discovery operation that identifies one or more causal relationships between the plurality of clinical metrics. The processing system 1620 may further execute the causal inferencing SW module 1636 to generate one or more inferences associated with the medical system based at least in part on the DAG.

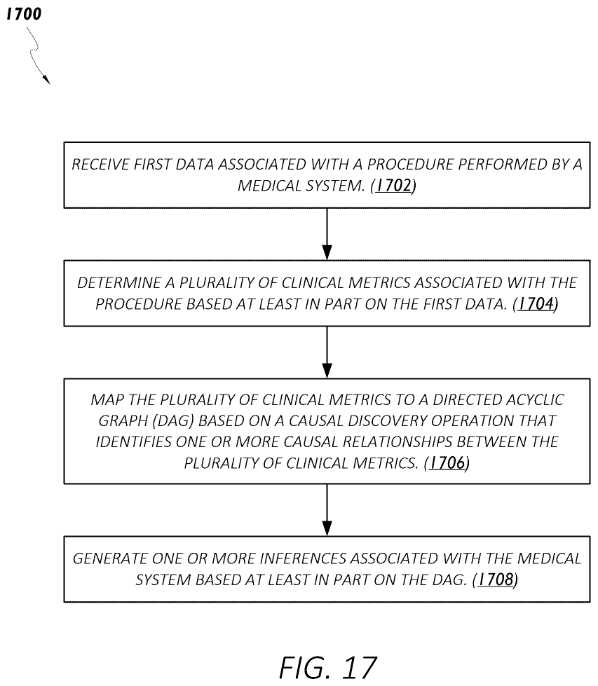

FIG. 17 shows an illustrative flowchart depicting an example causal inferencing operation 1700, according to some implementations. In some implementations, the example operation 1700 may be performed by a causal inferencing system such as the causal inferencing system 1600 of FIG. 16 or the inferencing system 900 of FIG. 9.

The causal inferencing system receives first data associated with a procedure performed by a medical system (1702). In some aspects, the medical system may include a robotic system and the first data may include telemetry generated by the robotic system. In some implementations, the telemetry indicates one or more poses, movements, or actuations of a robotic apparatus associated with the robotic system. In some other implementations, the telemetry indicates one or more poses of an instrument or scope associated with the robotic system. The causal inferencing system determines a plurality of clinical metrics associated with the procedure based at least in part on the first data (1704). The causal inferencing system maps the plurality of clinical metrics to a directed acyclic graph (DAG) based on a causal discovery operation that identifies one or more causal relationships between the plurality of clinical metrics (1706). The causal inferencing system further generates one or more inferences associated with the medical system based at least in part on the DAG (1708).

In some aspects, the determining of the plurality of clinical metrics may include determining one or more values for each clinical metric of the plurality of clinical metrics based at least in part on the first data, detecting one or more outliers among the one or more values for each clinical metric, and transforming each of the one or more outliers into a maximum value or a minimum value associated with a normalized dataset. In some implementations, the determining of the one or more values for each clinical metric of the plurality of clinical metrics may include imputing a value for a first clinical metric of the plurality of clinical metrics based at least in part on one or more other values associated with the first clinical metric or one or more values associated with a second clinical metric of the plurality of clinical metrics. In some implementations, the imputed value may represent a mean, median, or mode of the one or more other values associated with the first clinical metric.

In some implementations, the causal inferencing system may further scale the one or more values for each clinical metric of the plurality of clinical metrics so that each of the one or more values is greater than or equal to 0 and less than or equal to 1. In some other implementations, the causal inferencing system may determine an arithmetic mean of the one or more values for each clinical metric of the plurality of clinical metrics and scale the one or more values for each clinical metric of the plurality of clinical metrics to unit variance based at least in part on the arithmetic mean.

In some aspects, the casual inferencing system may further determine a subset of clinical metrics, of the plurality of clinical metrics, that have a causal effect on the procedure being successful or unsuccessful and determine a range of values for each clinical metric in the subset of clinical metrics that causes the procedure to be successful. In some implementations, the generating of the one or more inferences may include receiving second data associated with the procedure performed by the medical system, determining a respective value for each clinical metric in the subset of clinical metrics based at least in part on the second telemetry data, and identifying one or more clinical metrics in the subset of clinical metrics for which the determined value is outside the range of values that causes the procedure to be successful.

In some aspects, the causal inferencing system may further determine a graphical causal model (GCM) based on the DAG and data generation information associated with the plurality of clinical metrics. In such aspects, the one or more inferences may be further generated based on the GCM. In some implementations, the generating of the one or more inferences may include receiving a complaint indicating a problem associated with the medical system and identifying one or more clinical metrics of the plurality of clinical metrics as a root cause of the problem. In some other implementations, the one or more inferences may include a simulated randomized controlled trial (RCT).

Those of skill in the art will appreciate that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative logics, logical blocks, modules, circuits and algorithm processes described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and processes described herein. Whether such functionality is implemented in hardware or software depends upon the particular application and design constraints imposed on the overall system.

In the foregoing specification, implementations have been described with reference to specific examples thereof. It will, however, be evident that various modifications and changes may be made thereto without departing from the broader scope of the disclosure as set forth in the appended claims. The specification and drawings are, accordingly, to be regarded in an illustrative sense rather than a restrictive sense.

As used herein, a phrase referring to “at least one of” a list of items refers to any combination of those items, including single members. As an example, “at least one of: a, b, or c” is intended to cover: a, b, c, a-b, a-c, b-c, and a-b-c.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein.

What is claimed is:

1. A computer-implemented method for causal inferencing, comprising:
receiving first data associated with a procedure performed by a medical system;
determining a plurality of clinical metrics associated with the procedure based at least in part on the first data;
mapping the plurality of clinical metrics to a directed acyclic graph (DAG) based on a causal discovery operation that identifies one or more causal relationships between the plurality of clinical metrics; and
generating one or more inferences associated with the medical system based at least in part on the DAG.

2. The method of claim 1, wherein the medical system comprises a robotic system and the first data includes telemetry generated by the robotic system.

3. The method of claim 2, wherein the telemetry indicates one or more poses, movements, or actuations of a robotic apparatus associated with the robotic system.

4. The method of claim 2, wherein the telemetry indicates one or more poses of an instrument or scope associated with the robotic system.

5. The method of claim 1, wherein the determining of the plurality of clinical metrics comprises:
determining one or more values for each clinical metric of the plurality of clinical metrics based at least in part on the first data;
detecting one or more outliers among the one or more values for each clinical metric; and
transforming each of the one or more outliers into a maximum value or a minimum value associated with a normalized dataset.

6. The method of claim 5, wherein the determining of the one or more values for each clinical metric of the plurality of clinical metrics comprises:
imputing a value for a first clinical metric of the plurality of clinical metrics based at least in part on one or more other values associated with the first clinical metric or one or more values associated with a second clinical metric of the plurality of clinical metrics.

7. The method of claim 6, wherein the imputed value represents a mean, median, or mode of the one or more other values associated with the first clinical metric.

8. The method of claim 5, further comprising:
scaling the one or more values for each clinical metric of the plurality of clinical metrics so that each of the one or more values is greater than or equal to 0 and less than or equal to 1.

9. The method of claim 5, further comprising:
determining an arithmetic mean of the one or more values for each clinical metric of the plurality of clinical metrics; and
scaling the one or more values for each clinical metric of the plurality of clinical metrics to unit variance based at least in part on the arithmetic mean.

10. The method of claim 1, further comprising:
determining a graphical causal model (GCM) based on the DAG and data generation information associated with the plurality of clinical metrics, the one or more inferences further being generated based on the GCM.

11. The method of claim 1, further comprising:
determining a subset of clinical metrics, of the plurality of clinical metrics, that have a causal effect on the procedure being successful or unsuccessful; and
determining a range of values for each clinical metric in the subset of clinical metrics that causes the procedure to be successful.

12. The method of claim 11, wherein the generating of the one or more inferences comprises:
receiving second data associated with the procedure performed by the medical system;
determining a respective value for each clinical metric in the subset of clinical metrics based at least in part on the second telemetry data; and
identifying one or more clinical metrics in the subset of clinical metrics for which the determined value is outside the range of values that causes the procedure to be successful.

13. The method of claim 1, wherein the generating of the one or more inferences comprises:
receiving a complaint indicating a problem associated with the medical system; and
identifying one or more clinical metrics of the plurality of clinical metrics as a root cause of the problem.

14. The method of claim 1, wherein the one or more inferences include a simulated randomized controlled trial (RCT).

15. A causal inferencing system comprising:
a processing system; and
a memory storing instructions that, when executed by the processing system, cause the causal inferencing system to:
receive first data associated with a procedure performed by a medical system;
determine a plurality of clinical metrics associated with the procedure based at least in part on the first data;
map the plurality of clinical metrics to a directed acyclic graph (DAG) based on a causal discovery operation that identifies one or more causal relationships between the plurality of clinical metrics; and
generate one or more inferences associated with the medical system based at least in part on the DAG.

16. The causal inferencing system of claim 15, wherein the medical system comprises a robotic system and the first data includes telemetry generated by the robotic system.

17. The causal inferencing system of claim 16, wherein the telemetry indicates one or more poses, movements, or actuations of a robotic apparatus associated with the robotic system.

18. The causal inferencing system of claim 16, wherein the telemetry indicates one or more poses of an instrument or scope associated with the robotic system.

19. The causal inferencing system of claim 15, wherein the generating of the one or more inferences comprises:
determining a subset of clinical metrics, of the plurality of clinical metrics, that have a causal effect on the procedure being successful or unsuccessful;
determining a range of values for each clinical metric in the subset of clinical metrics that causes the procedure to be successful;
receiving second data associated with the procedure performed by the medical system;
determining a respective value for each clinical metric in the subset of clinical metrics based at least in part on the second telemetry data; and
identifying one or more clinical metrics in the subset of clinical metrics for which the determined value is outside the range of values that causes the procedure to be successful.

20. The causal inferencing system of claim 15, wherein the generating of the one or more inferences comprises:
receiving a complaint indicating a problem associated with the medical system; and
identifying one or more clinical metrics of the plurality of clinical metrics as a root cause of the problem.
